(19) United States
(12) Patent Application Publication
(10) Pub. No.: US 2017/0182090 A1
(43) Pub. Date: Jun. 29, 2017

(54) Sulfate Salt Solution Laxative Compositions and Methods of Use Thereof

(71) Applicant: Braintree Laboratories, Inc., Braintree, MA (US)

(72) Inventors: Mark vB Cleveland, Norwell, MA (US); John S. Fordtran, Dallas, TX (US); Edmund V. Dennett, South Walpole, MA (US)

(21) Appl. No.: 15/456,497

(22) Filed: Mar. 11, 2017

Related U.S. Application Data
(63) Continuation-in-part of application No. PCT/US15/49571, filed on Sep. 11, 2015.
(60) Provisional application No. 62/049,830, filed on Sep. 12, 2014.

(51) Int. Cl. A61K 33/04; A61K 9/00

(52) U.S. Cl. CPC A61K 33/04; A61K 9/0095

(57) ABSTRACT

The present invention is generally directed to an oral pharmaceutical composition comprising a sulfate salt, for example, sodium sulfate and/or potassium sulfate, wherein the composition is substantially free of magnesium sulfate. The present invention is further directed to use of such oral pharmaceutical compositions to induce laxation or to treat or prevent constipation, e.g., opioid-induced constipation.

### FIELD OF THE INVENTION

This disclosure relates generally to the field of medicine and particularly to treatment of disease associated with the gastrointestinal tract.

### BACKGROUND OF THE INVENTION

### Laxative Formulations

Laxatives are generally categorized as osmotic, saline or stimulant. Commonly used osmotic (polyethylene glycol, lactulose) and saline laxatives (sodium phosphate, magnesium citrate) often are slow to act taking as long as several days to induce a bowel movement (Lembo et al., N Engl J Med (2003) 349:1360-1368). Saline laxatives typically have been associated with potentially dangerous electrolyte abnormalities (DiPalma, Rev Gastroenterol Disord (2004) 4(suppl 2): S34-S42). Cathartic laxatives (bisacodyl, senna) often take as long as six hours to have an effect and have been associated with abdominal cramping and, occasionally, serious adverse events such as ischemic colitis (DiPalma, Rev Gastroenterol Disord (2004) 4(suppl 2):S34-S42; Arhan et al., Amer J Gastroenterol (2009) 104:250-251).

Sodium phosphate preparations (i.e., phospho-soda), consisting primarily of monobasic sodium phosphate monohydrate and dibasic sodium phosphate heptahydrate, may be used for bowel cleansing for colonoscopy, or other procedures, or as a laxative. However, such preparations induce substantial adsorption of phosphate ions resulting in serum hypokalemia, hyperphosphatemia, increased calcium-phosphate and associated reductions in serum calcium levels. In some instances, use of phospho-soda preparations can lead to acute phosphate nephropathy.

### Drug-Induced Constipation

Constipation is an acute or chronic condition in which bowel movements occur less often than usual or consist of hard, dry stools that are painful or difficult to pass. Diagnostic criteria for functional constipation are defined by the ROME III criteria (Longstreth et al., Gastroenterol (2006) 130(5):1480-1491). Constipation may result from the administration of a variety of different medications including prescription drugs such as opioids, anticholinergic agents, tricyclic antidepressants (e.g., amytriptyline, nortriptyline), calcium channel blockers (e.g., verapamil), antiparkinsonian drugs, sympathomimetics (e.g., ephedrine, terbutaline), antipsychotics (e.g., chlorpromazine), diuretics (e.g., furosemide) and antihistamines (e.g., diphenyldramine) as well as over the counter drugs such as antacids, calcium supplements, iron, antidiarrheal agents (e.g., loperamide, attapulgite) and nonsteroidal anti-inflammatory drugs (e.g., ibuprofen) (Toney et al., Pract Gastroenterol (2008) May: 12-28). Opioid induced constipation results from both central and peripheral effects including delayed gastric emptying by producing gastroparesis, increased transit time of stool through the small intestine and colon by decreasing propulsive peristalsis, decreased urge to defecate and interference with fluid and electrolyte regulation (McMillan et al., Cancer Control (2004) 11(3, Supplement 1):3-9). Chronic administration of opioids for pain management is a frequent cause of constipation in cancer patients. For instance, 40% to 64% of hospice patients with cancer have been found to have opioid-induced constipation (McMillan et al., Cancer Control (2004) 11(3, Supplement 1):3-9).

Accordingly, there is a need in the art for a fast-acting laxative formulation that maintains the electrolyte balance in the patient. In particular, there is a need in the art for a fast-acting laxative to treat patients with drug-induced constipation, e.g., opioid-induced constipation, without disturbing the electrolyte balance in the patient.

### SUMMARY OF INVENTION

The present invention is based, at least in part, on the identification of a unique, fast-acting pharmaceutical composition comprising a low dose of sulfate salts, for example, sodium sulfate and potassium sulfate, for inducing bowel movements and/or the treatment or prevention of constipation, e.g., drug-induced constipation, such as opioid-induced constipation in a subject.

An advantage of the disclosed compositions is their safety. In particular, the disclosed compositions also do not cause clinically significant electrolyte shifts. Due to the lack of clinically significant electrolyte shifts, subjects can take the disclosed formulations without suffering from additional complications associated with the treatment of constipation induced by other medications. It should be noted that the present compositions, formulations, and methods allow for treatment of conditions such as fecal retention, constipation, and hard stools by providing a formulation that can be used without significant adverse events.

Accordingly, in one aspect, the present invention provides oral pharmaceutical compositions, e.g., tablet, powder, or liquid compositions, for inducing a bowel movement in a human subject. The compositions include sodium sulfate and potassium sulfate, and are substantially free of magnesium sulfate.

In another aspect, the present invention provides oral pharmaceutical compositions, e.g., tablet, powder, or liquid compositions, for treating constipation in a human subject. The compositions include sodium sulfate and potassium sulfate, and are substantially free of magnesium sulfate.

The compositions of the invention are suitable for administration to the human subject by direct oral ingestion; suitable for administration to the human subject by disintegration in an aqueous solution prior to oral ingestion; or suitable for both administration to the human subject by direct oral ingestion and by disintegration in an aqueous solution prior to oral ingestion.

In one embodiment, the compositions do not produce a clinically significant electrolyte shift, e.g., a clinically significant sodium and potassium shift, upon administration to the human subject.

In one embodiment, the compositions are substantially free of polyethylene glycol (PEG). In another embodiment, the compositions are substantially free of phosphates.

In one embodiment, the compositions induce a bowel movement in a human subject within 5 hours of administration. In another embodiment, the compositions induce a bowel movement in a human subject within 4 hours of administration. In yet another embodiment, the compositions induce a bowel movement in a human subject within 3 hours of administration. In another embodiment, the compositions induce a bowel movement in a human subject within 2 hours of administration.

In one embodiment, the compositions induce 1 to 6 bowel movements in a human subject within 24 hours of administration. In another embodiment, the compositions induce 1 to 3 bowel movements in a human subject within 24 hours of administration. In yet another embodiment, the compositions induce 1 to 3 bowel movements in a human subject within 24 hours of administration.

In one embodiment, the compositions induce a bowel movement in a human subject with a Bristol Stool Scale rating of 4 to 7.

In one embodiment, the compositions comprise about 30 mmol to about 100 mmol of sulfate salts. In another embodiment, the compositions comprise about 60 mmol to about 80 mmol of sulfate salts. In yet another embodiment, the compositions comprise about 50 mmol of sulfate salts. In another embodiment, the compositions comprise about 60 mmol of sulfate salts. In a further embodiment, the compositions comprise about 80 mmol of sulfate salts.

In one embodiment, the sodium sulfate and potassium sulfate are present in the composition at a ratio of about 2:1 to about 4:1. In another embodiment, the sodium sulfate and potassium sulfate are present in the composition at a ratio of about 2.5:1 to about 3.5:1. In yet another embodiment, the sodium sulfate and potassium sulfate are present in the composition at a ratio of about 3:1.

In one embodiment, the composition comprises about 3 g to about 10 g of sodium sulfate. In another embodiment, the composition comprises about 5 g to about 8 g of sodium sulfate.

In one embodiment, the composition comprises about 1 g to about 8 g of potassium sulfate. In another embodiment, the composition comprises about 2 g to about 4 g of potassium sulfate.

In one embodiment, the composition comprises about 3 g to about 10 g of total sulfate. In another embodiment, the composition comprises about 4 g of total sulfate. In another embodiment, the composition comprises about 5 g of total sulfate. In yet another embodiment, the composition comprises about 6 g of total sulfate. In another embodiment, the composition comprises about 7 g of total sulfate.

In one embodiment, the composition comprises at least one excipient selected from the group consisting of a disintegrant, a binder, a glidant, a lubricant, a flavor, a preservative, a sweetener, and combinations thereof.

In a further aspect, the present invention provides methods for inducing a bowel movement in a human subject. The methods include orally administering to the subject a pharmaceutical composition comprising a 3:1 ratio of sodium sulfate:potassium sulfate, wherein the composition is substantially free of magnesium sulfate, and wherein the composition does not produce a clinically significant electrolyte shift upon administration to the human subject, thereby inducing a bowel movement in the subject.

In one embodiment, the composition is administered to the human subject in a single dose.

In one embodiment, the single dose is administered once per day. In another embodiment, the single dose is administered twice per day.

In one embodiment, the single dose is administered once per day for 1 or more days. In another embodiment, the single dose is administered once per day for up to 28 days.

In one embodiment, the composition is administered to the subject in a single dose on each of 28 days.

In one embodiment, the composition induces 3 or more bowel movements within any 7 consecutive days of treatment.

In one embodiment, the composition induces a bowel movement in the human subject within 3 hours of administration. In another embodiment, the composition induces a bowel movement in the human subject within 2 hours of administration.

In one embodiment, the composition induces 1 to 6 bowel movements in a human subject within 24 hours of administration.

In another aspect, the present invention provides methods for inducing a bowel movement in a human subject. The methods include orally administering to the subject a composition of the invention, and allowing the composition to induce a bowel movement in the subject, thereby inducing a bowel movement in the human subject.

In yet another aspect, the present invention provide smethods for treating or preventing constipation in a human subject. The methods include orally administering to the subject a composition of the invention, in an amount effective to treat or prevent constipation in a human subject, and thereby treating or preventing constipation in the human subject.

In one embodiment, the composition induces a bowel movement in the human subject within 5 hours of administration. In another embodiment, n the composition induces a bowel movement in the human subject within 4 hours of administration. In yet another embodiment, the composition induces a bowel movement in the human subject within 3 hours of administration.

In one embodiment, the human subject no longer meets the ROME III criteria following administration of the composition for 7 consecutive days.

In one embodiment, the composition induces a bowel movement in the human subject with a Bristol Stool Scale rating of 4 to 7.

In one embodiment, the human subject does not experience a clinically significant electrolyte shift, e.g., a clinically significant sodium and potassium shift.

In one embodiment, the human subject suffers from drug-induced constipation, e.g., opioid-induced constipation. In one embodiment, the human subject is receiving opioids chronically. In one embodiment, the human subject is a cancer patient.

As disclosed herein, the formulations induce a laxative effect (e.g., laxation) in a subject. As used herein, the term “a” means one or more unless specifically defined otherwise. In certain embodiments, the formulation comprises a dose of from about 0.9 grams of sodium sulfate to about 8.0 grams of sodium sulfate and from about 0.3 grams to about 3.5 grams of potassium sulfate. In other embodiments, the formulation comprises a dose of from about 5.0 grams to about 7.0 grams of sodium sulfate and from about 2.0 grams to about 2.5 grams of potassium sulfate. In other embodiments, the formulation comprises a dose of about 5.5 grams of sodium sulfate and about 2.7 grams of potassium sulfate. In more particular embodiments, the formulation comprises a dose of 5.54 grams of sodium sulfate and 2.27 grams of potassium sulfate. In some embodiments, the dose can comprise from about 0.5 grams to about 1.5 grams of sodium sulfate and from about 0.2 grams to about 0.6 grams of potassium sulfate. In other embodiments, the dose comprises from about 0.9 grams of sodium sulfate and about 0.4 grams of potassium sulfate. In some embodiments, each dose comprises about 0.6 grams to about 1.4 grams of sodium sulfate or alternatively from about 0.7 grams to about 1.3 grams of sodium sulfate. Each dose can comprise from about 0.8 grams to about 1.2 grams out 0.8 grams to about 1.1 grams or from about 0.8 grams to about 1.0 grams of sodium sulfate. Each dose can further comprise from about 0.3 grams of potassium sulfate to about 0.5 grams of potassium sulfate. An exemplary dose comprises 0.9233 grams of sodium sulfate and 0.37833 of potassium sulfate. As used herein, the term “about” means within +/−10% of the recited value. For instance, about 2.0 would cover from 1.8 to 2.2.

In certain embodiments, the formulation is a solid oral dosage form. In more embodiments, the formulation is a solid oral dosage form. In some embodiments, the solid oral dosage form is a tablet, capsule, compressed capsule, gel capsule, or caplet. In particular embodiments, the solid oral dosage form is a tablet. In very specific embodiments, an oral pharmaceutical tablet composition, wherein the composition is capable of administration by direct oral ingestion and by disintegration in water prior to oral ingestion.

In certain embodiments, the formulation comprises one or more tablets to be administered to a patient. For instance, the formulation can comprise multiple tablets administered. In other embodiments, the formulation comprises from about 1 to about 10 tablets. In still other embodiments, the formulation comprises from about 1 to about 6 tablets.

In some embodiments, each solid oral dosage form comprises from about 0.5 grams to about 1.5 grams of sodium sulfate and from about 0.2 grams to about 0.6 grams of potassium sulfate. In specific embodiments, the formulation comprises from about 0.9 grams of sodium sulfate and about 0.4 grams of potassium sulfate per solid oral dosage form. In very specific embodiments, the formulation comprises 0.9233 grams of sodium sulfate and 0.3783 grams of potassium sulfate per solid oral dosage form. A single solid oral dosage form can be a dose.

In other embodiments, each solid oral dosage form comprises about 0.6 grams to about 1.4 grams of sodium sulfate. In other embodiments, the solid oral dosage forms each comprise from about 0.7 grams to about 1.3 grams of sodium sulfate. In still other embodiments, each solid oral dosage form comprises from about 0.8 grams to about 1.2 grams of sodium sulfate. In yet other embodiments, each solid oral dosage form comprises from about 0.8 grams to about 1.1 grams or from about 0.8 grams to about 1.0 grams of sodium sulfate.

In particular embodiments, each solid oral dosage form comprises from about 0.3 grams to about 0.5 grams of potassium sulfate. In more particular embodiments, each solid oral dosage form comprise from about 0.8 grams to about 1.1 grams of sodium sulfate and 0.3 grams to about 0.5 grams of potassium sulfate.

In yet other embodiments, a dose of the formulation comprises from about 4.00 to about 7.00 grams of sodium sulfate and from about 1.00 to about 3.5.0 grams of potassium sulfate. In particular embodiments, the dose of the formulation comprises from about 5.00 grams to about 6.00 grams of sodium sulfate and from about 2.00 grams to 2.50 grams of potassium sulfate. In more particular embodiments, the dose of the formulation comprises 5.54 grams of sodium sulfate and 2.27 grams of potassium sulfate.

In still other embodiments, the formulation is a powder formulation comprising at least 6.0 grams of sulfate salts. In particular embodiments, the powder formulation comprises from about 4.0 grams of sodium sulfate to about 8.0 grams of sodium sulfate in combination with potassium sulfate in an amount from about 1.0 grams to about 3.5 grams. In more particular embodiments, the powder formulation comprises about 5.5 grams of sodium sulfate and 2.7 grams of potassium sulfate. In particular embodiments, the powder formulation comprises particular doses. For instance, each dose can comprise from about 0.5 grams to about 1.5 grams of sodium sulfate and from about 0.2 grams to about 0.6 grams of potassium sulfate. Each dose can also comprise from about 0.9 grams of sodium sulfate and about 0.4 grams of potassium sulfate. In some embodiments, each dose comprises about 0.6 grams to about 1.4 grams of sodium sulfate or alternatively from about 0.7 grams to about 1.3 grams of sodium sulfate. Each dose can comprise from about 0.8 grams to about 1.2 grams out 0.8 grams to about 1.1 grams or from about 0.8 grams to about 1.0 grams of sodium sulfate. Each dose can further comprise from about 0.3 grams of potassium sulfate to about 0.5 grams of potassium sulfate. An exemplary dose comprises 0.9233 grams of sodium sulfate and 0.37833 of potassium sulfate.

In certain embodiments, the ratio of sodium sulfate to potassium sulfate in the disclosed formulations is about 3 to 1. In other embodiments, the ratio of sodium sulfate to potassium sulfate in the disclosed formulations is about 2.5 to 1. In still other embodiments, the ratio of sodium sulfate to potassium sulfate is 2.44 to 1.

In particular embodiments, the formulation is a liquid. In particular embodiments, the powder formulation comprises from about 4.0 grams of sodium sulfate to about 8.0 grams of sodium sulfate in combination with potassium sulfate in an amount from about 1.0 grams to about 3.5 grams. In more particular embodiments, the powder formulation comprises about 5.5 grams of sodium sulfate and 2.7 grams of potassium sulfate.

In certain embodiments, the tablets exhibit the dissolution characteristics of not less than 75% (Q) in 15 minutes, in 30 minutes, or in one hour. In some embodiments, the tablets exhibit the dissolution characteristics of not less than (e.g., at least) 75% (Q) in 10 minutes. In other embodiments, the tablets exhibit the dissolution characteristics of not less than 80% (Q) in 10 minutes, in 15 minutes, in 30 minutes, or in one hour. In still other embodiments, the tablets exhibit the dissolution characteristics of not less than 90% (Q) in 10 minutes, in 15 minutes, in 30 minutes, or in one hour. In still more embodiments, the tablets exhibit the dissolution characteristics of at least 95% (Q) in 10 minutes, in 15 minutes, in 30 minutes, or in one hour.

In certain embodiments the oral pharmaceutical tablet composition disintegrates in water at about 2° C. or greater in less than about 150 seconds; disintegrates in water at about 8° C. or greater in less than about 90 seconds; disintegrates in water at about 5° C. or greater in less than about 120 seconds; disintegrates in water at about 2° C. or greater in less than about 150 seconds; does not disintegrate in the mouth in less than about 30 seconds upon direct oral ingestion; and/or does not disintegrate in the mouth in less than about 60 seconds upon direct oral ingestion.

In still more embodiments, the disclosed formulations do not cause clinically significant electrolyte shifts in the subject.

Further aspects include a method of treating constipation in a subject. The method comprises administering an effective amount of sodium sulfate and potassium sulfate. The method further comprises producing a laxative effect (i.e., causing a spontaneous bowel movement) in the subject. In certain embodiments, the effective amount of sodium sulfate and potassium sulfate causes the subject to have at least three spontaneous bowel movements. In certain embodiments, the subject has three spontaneous bowel movements in a seven day treatment period.

In particular embodiments, the formulation is administered at least once per day. In more particular embodiments, the formulation is administered on an as needed basis by the subject.

In still further embodiments, the formulation does not cause clinically significant electrolyte shifts in a subject. One of ordinary skill in the art will recognize that a clinically significant event must have a genuine, noticeable, and unexpected effect on the life of a subject. Mere changes in electrolyte levels outside of a given range in a single patient or small group of patients will not rise to the level of a clinically significant event. Rather, a clinically significant event is one that substantially effects the life of a subject. A clinically significant electrolyte shift can also be found when the mean electrolyte levels of a population shift outside of the normal range for the population of patients, not just in an individual patient or small group of patients within the population. Furthermore, one of ordinary skill will recognize that an event must be unexpected as well. Events that are expected when administering a laxative would not be clinically significant events due to their being expected in some individuals.

In some embodiments, the formulation further comprises one or more excipients. In other embodiments, the one or more excipients are selected from the group consisting of binders, lubricants, glidants, disintegrants, and combinations thereof.

### BRIEF DESCRIPTION OF DRAWINGS

FIG. 1 graphically depicts stool output in response to sulfate dose, as described in Example 1.

FIG. 2 graphically depicts stool water content in response to sulfate dose, as described in Example 1.

FIG. 3 depicts the screening and randomization of study subjects as described in Example 2.

### DETAILED DESCRIPTION OF THE INVENTION

Laxatives are not typically formulated to maintain an electrolyte balance when administered to a subject. As a result, currently available laxatives typically lead to clinically significant electrolyte shifts as a result of, for example, sodium absorption and loss of potassium. The present invention is based, at least in part, on the identification of a unique, fast-acting pharmaceutical composition comprising a low dose of sulfate salts, for example, sodium sulfate and potassium sulfate, for inducing bowel movements and/or the treatment or prevention of constipation, e.g., drug-induced constipation, such as opioid-induced constipation, which do not produce a clinically significant electrolyte shift, e.g., a clinically significant sodium and/or potassium shift, in the subject to whom the pharmaceutical composition is administered. Indeed, the present invention is based, at least in part, on the identification of a pharmaceutical composition that provides on demand relief for patients suffering from constipation. Moreover, by providing an oral pharmaceutical composition, e.g., a pharmaceutical composition for direct oral ingestion or for disintegration in water prior to oral ingestion, the present invention further offers a convenient and easy to use formulation.

### I. Pharmaceutical Compositions

In one aspect, the present invention provides an oral pharmaceutical composition for inducing a bowel movement in a subject, the composition comprising a combination of sulfate salts, for example, sodium sulfate and potassium sulfate, wherein the composition is substantially free of magnesium sulfate. In another aspect, the present invention provides an oral pharmaceutical composition for treating constipation in a subject, the composition comprising a combination of sulfate salts, for example, sodium sulfate and potassium sulfate, wherein the composition is substantially free of magnesium sulfate. In some embodiments of the invention, the oral pharmaceutical compositions are tablet compositions which are suitable for administration to a subject by direct oral ingestion. In other embodiments, the oral pharmaceutical compositions are tablets which are suitable for disintegration in water prior to oral ingestion. In yet other embodiments, oral pharmaceutical compositions of the invention are tablet compositions which are suitable for administration by both direct oral ingestion and disintegration in water prior to oral ingestion. In further embodiments of the invention, the oral pharmaceutical compositions are aqueous solutions.

### Sulfate Salts

As used herein, the term “sulfate salt” refers to at least one combination of sulfate ion, i.e., SO₄²⁻, and an appropriate cation. Non-limiting examples of sulfate salts for use in the present invention include, for example, sodium sulfate (Na₂SO₄) and potassium sulfate (K₂SO₄) and combinations thereof.

In a particular embodiment, the oral pharmaceutical composition includes sodium sulfate and potassium sulfate. In certain embodiments, the oral pharmaceutical compositions of the invention comprise an aqueous solution of at least two sulfate salts, e.g., sodium sulfate and potassium sulfate. For purposes of the present invention, it is understood that when present in solution, the sulfate salt may take the form of the respective ions, i.e., sulfate and sodium and potassium. Accordingly, as used herein, the term “sulfate salt” encompasses embodiments wherein the ions are present in salt or ion form.

In certain embodiments, the oral pharmaceutical composition includes at least about 20 mmol, 25 mmol, 30 mmol, 35 mmol, 40 mmol, 45 mmol, 50 mmol, 55 mmol, 60 mmol, 65 mmol, 70 mmol, 75 mmol, 80 mmol, 85 mmol, 90 mmol, 95 mmol, 100 mmol, 110 mmol, 120 mmol, 130 mmol, 140 mmol or 150 mmol of sulfate salts as the active ingredient. Ranges within one or more of the preceding values, e.g., 20-30 mmol, 20-40 mmol, 20-50 mmol, 20-60 mmol, 20-70 mmol, 20-80 mmol, 20-90 mmol, 20-100 mmol, 20-110 mmol, 20-120 mmol, 20-130 mmol, 20-140 mmol, 20-150 mmol, 30-40 mmol, 30-50 mmol, 30-60 mmol, 30-70 mmol, 30-80 mmol, 30-90 mmol, 30-100 mmol, 30-110 mmol, 30-120 mmol, 30-130 mmol, 30-140 mmol, 30-150 mmol, 40-50 mmol, 40-60 mmol, 40-70 mmol, 40-80 mmol, 40-90 mmol, 40-100 mmol, 40-110 mmol, 40-120 mmol, 40-130 mmol, 40-140 mmol, 40-150 mmol, 50-60 mmol, 50-70 mmol, 50-80 mmol, 50-90 mmol, 50-100 mmol, 50-110 mmol, 50-120 mmol, 50-130 mmol, 50-140 mmol, 50-150 mmol, 60-70 mmol, 60-80 mmol, 60-90 mmol, 60-100 mmol, 60-110 mmol, 60-120 mmol, 60-130 mmol, 60-140 mmol, 60-150 mmol, 70-80 mmol, 70-90 mmol, 70-100 mmol, 70-110 mmol, 70-120 mmol, 70-130 mmol, 70-140 mmol and 70-150 mmol are contemplated by the invention. In a specific embodiment of the invention, the oral pharmaceutical composition includes at least about 60 mmol of a sulfate salt consisting of sodium sulfate and potassium sulfate. In a further embodiment of the invention, the oral pharmaceutical composition includes at least about 80 mmol of a sulfate salt consisting of sodium sulfate and potassium sulfate.

In certain embodiments, the oral pharmaceutical composition comprises at least about 1 gram, 2 grams, 3 grams, 4 grams, 5 grams, 6 grams, 7 grams, 8 grams, 9 grams, 10 grams, 11 grams, 12 grams, 13 grams, 14 grams or 15 grams of sodium sulfate. Ranges within one or more of the preceding values, e.g., 1-3 grams, 1-5 grams, 1-8 grams, 1-10 grams, 1-12 grams, 1-15 grams, 3-5 grams, 3-8 grams, 3-10 grams, 3-12 grams, 3-15 grams, 5-8 grams, 5-10 grams, 5-12 grams, 5-15 grams, 8-10 grams, 8-12 grams and 8-15 grams of sodium sulfate are contemplated by the invention. In yet another embodiment, the oral pharmaceutical composition comprises about 4 grams or about 5 grams of sodium sulfate.

In certain embodiments, the oral pharmaceutical composition comprises at least about 1 gram, 2 grams, 3 grams, 4 grams, 5 grams, 6 grams, 7 grams, 8 grams, 9 grams, 10 grams, 11 grams, 12 grams, 13 grams, 14 grams or 15 grams of potassium sulfate. Ranges within one or more of the preceding values, e.g., 1-3 grams, 1-5 grams, 1-8 grams, 1-10 grams, 1-12 grams, 1-15 grams, 3-5 grams, 3-8 grams, 3-10 grams, 3-12 grams, 3-15 grams, 5-8 grams, 5-10 grams, 5-12 grams, 5-15 grams, 8-10 grams, 8-12 grams and 8-15 grams of potassium sulfate are contemplated by the invention. In particular embodiments, the oral pharmaceutical composition comprises about 1 gram or about 2 grams of potassium sulfate.

In certain embodiments, the oral pharmaceutical composition comprises at least about 1 gram, 2 grams, 3 grams, 4 grams, 5 grams, 6 grams, 7 grams, 8 grams, 9 grams 10 grams, 11 grams, 12 grams, 13 grams, 14 grams, 15 grams, 16 grams, 17 grams, 18 grams, 19 grams or 20 grams of total sulfate. Ranges within one or more of the preceding values, e.g., 1-3 grams, 1-5 grams, 1-8 grams, 1-10 grams, 1-12 grams, 1-15 grams, 1-18 grams, 1-20 grams, 3-5 grams, 3-8 grams, 3-10 grams, 3-12 grams, 3-15 grams, 3-18 grams, 3-20 grams, 5-8 grams, 5-10 grams, 5-12 grams, 5-15 grams, 5-18 grams, 5-20 grams, 8-10 grams, 8-12 grams, 8-15, 8-18 grams, 8-20 grams, 10-12 grams, 10-15 grams, 10-18 grams and 10-120 grams of total sulfate are contemplated by the invention. In a particular embodiment, the oral pharmaceutical composition comprises about 4 grams of total sulfate. In another embodiment, the oral pharmaceutical composition comprises about 5 grams of total sulfate. In yet another embodiment, the oral pharmaceutical composition comprises about 6 grams of total sulfate. In a further embodiment, the oral pharmaceutical composition comprises about 7 grams of total sulfate.

In certain embodiments, the oral pharmaceutical composition includes sodium sulfate and potassium sulfate which are present in the composition at a ratio of about 1:1, 1.5:1, 2:1, 2.5:1, 3:1, 3.5:1, 4:1, 4.5:1, or 5:1. In another embodiment, the oral pharmaceutical composition includes sodium sulfate and potassium sulfate present at a ratio of about 2:1 to about 4:1 or about 2.5:1 to about 3.5:1. In particular embodiments, the oral pharmaceutical composition includes sodium sulfate and potassium sulfate present at a ratio of about 1:1 or about 1:3.

In a particular embodiment, the composition is substantially free of phosphate (PO₄³⁻) salts, present as a salt or in ion form. In a further embodiment, the composition is substantially free of polyethylene glycol (PEG). In a particular embodiment, the oral pharmaceutical composition is substantially free of magnesium sulfate. As used herein, the term “substantially free” refers to the complete absence of, or insignificant presence of, a particular element, e.g., phosphate salts, magnesium sulfate and/or PEG. In a particular embodiment, “substantially free” refers to oral pharmaceutical compositions that do not depend on the presence of such element, e.g., phosphate salts, magnesium sulfate and/or PEG, to achieve or enhance the desired laxative effect of the formulation and are, thus, understood to be substantially free of such element for the purposes described herein. As used herein, it is envisioned that minor amounts of such elements, e.g., phosphate salts, magnesium sulfate and/or PEG, may be present as inert ingredients, e.g., as binders, in certain formulations.

### Formulations

The oral pharmaceutical compositions of the invention may be provided in the form of an aqueous solution for oral ingestion or in tablet form, in particular in a tablet form suitable for both administration by direct oral ingestion and by disintegration in water prior to oral ingestion.

In one embodiment, the oral pharmaceutical compositions of the invention comprise a 3:1 ratio of sodium sulfate:potassium sulfate, are substantially free of magnesium sulfate, and do not produce a clinically significant electrolyte shift upon administration to a human subject.

The oral pharmaceutical compositions of the invention may be administered in a single dose, e.g., a single dose of a pharmaceutical composition comprising a 3:1 ratio of sodium sulfate:potassium sulfate, which is substantially free of magnesium sulfate, and does not produce a clinically significant electrolyte shift upon administration to a human subject. As used herein, a “single dose” refers to a physically discrete unit of inventive composition for administration to a subject to be treated. The single dose may be administered one or more times per day. In a particular embodiment, the single dose is administered once per day. In another embodiment, the single dose is administered twice per day. For example, a single dose may be split into two doses and each half of the split dose is administered to a subject at a different time during the day. The single dose may be administered once per day for 1 or more days. For example, the single dose may be administered once per day for up to 28 days. In particular embodiments, the single dose is administered for 1, 2, 3, 4, 5, 6, or 7 consecutive or non-consecutive days.

The oral pharmaceutical compositions of the invention induce a bowel movement in a subject following administration. The bowel movement may be produced in 1 to 24 hours following administration of the composition. For example, the bowel movement may be produced in 1, 2, 3, 4 or 5 hours following administration of the composition. In a particular embodiment, the oral pharmaceutical composition produces a bowel movement within 3 hours following administration. The composition may also induce 1 to 6 bowel movements within 24 hours of administration. For example, the composition may induce 2, 3, 4, or 5 bowel movements within 24 hours administration. The composition may also induce 1 to 3 complete bowel movements within 24 hours of administration.

In another embodiment of the invention, the composition induces a bowel movement in a subject with a Bristol Stool Scale rating of 4 to 7. As used herein, the “Bristol Stool Scale” refers a scale for rating the bowel movement as follows: 1=separate hard lumps like nuts (difficult to pass); 2=sausage shaped but lumpy; 3=like a sausage but with cracks on its surface; 4=like a sausage or snake, smooth and soft; 5=soft blobs with clear-cut edges (passed easily); 6=fluffy pieces with ragged edges, a mushy stool; 7=watery, no solid pieces, entirely liquid (O'Donnell et al., British Med J (1990) 300:439-440).

A. Aqueous Formulations

In one embodiment, the oral pharmaceutical compositions of the invention are aqueous formulations suitable for oral ingestion. Alternatively, the compositions of the invention may be administered as an aqueous solution delivered through a feeding tube.

The aqueous compositions may be reconstituted in water at varying temperatures. In alternative embodiments, the compositions of the invention may be dissolved in other aqueous fluids, for example, flavored drinks, and the subsequent aqueous dispersion ingested orally. For purposes of the present invention, saliva and components thereof are not considered an aqueous fluid.

In certain embodiments, the oral pharmaceutical composition is an aqueous solution with a volume of about 25 ml, 50 ml, 75 ml, 100 ml, 125 ml, 150 ml, 175 ml, 200 ml, 225 ml or 250 ml. Ranges within one or more of the preceding values, e.g., 25-50 ml, 25-75 ml, 25-100 ml, 25-150 ml, 25-200 ml, 25-250 ml, 50-75 ml, 50-100 ml, 50-150 ml, 50-200 ml, 50-250 ml, 75-100 ml, 75-150 ml, 75-200 ml, 75-250 ml, 100-150 ml, 100-200 ml and 100-250 ml are contemplated by the invention. In particular embodiments, the oral pharmaceutical composition is an aqueous solution with a volume of about 75 ml or about 100 ml.

B. Tablet Formulations

In one embodiment, the oral pharmaceutical compositions of the invention are provided in a tablet form. The tablet compositions of the invention may be suitable for administration by direct oral ingestion; or suitable for administration by disintegration in water prior to oral ingestion; or suitable for both administration by direct oral ingestion and disintegration in water prior to oral ingestion.

In one embodiment, the oral pharmaceutical compositions of the invention are provided in a tablet form as described in PCT/US2014/028805, filed Mar. 14, 2014, the entire contents of which are incorporated herein by reference.

The tablet compositions of the present invention are designed so as to maximize the sulfate content and to minimize the number of tablets required, both to induce the desired laxative effect and, further, to address patient compliance and convenience issues.

Accordingly, in one embodiment, the tablet composition is between about 1000 mg and about 3000 mg, between about 1500 mg and about 2500 mg, or about 1700 mg and about 2000 mg. In a particular embodiment, the tablet composition may be about 1800 mg.

In a preferred embodiment, the tablets have a hardness between about 7 kp to about 15 kp, about 7 kp to about 12 kp, about 8 kp to about 12 kp or about 10 kp to about 15 kp. The tablet should be of a sufficient hardness to allow for coating thereof and to allow for convenient manufacturing thereof, but not too hard so as to impede the desired disintegration profile in water.

The tablet may be of any shape. In one embodiment, the tablet is an oblong, biconvex tablet, for example, of about 1800 mg, with flat sides, optionally produced using standard compression tooling. This shape and size is convenient for patients because the oval shape with rounded faces provides for better oral administration.

The tablet compositions of the invention overcome challenges of patient compliance by providing a versatile composition that can be taken in a form convenient to the patient. In addition, the tablet compositions of the present invention overcome the challenges of patient compliance arising from the extreme hydroscopic properties of sodium sulfate, in part, by providing a tablet that can be disintegrated in water and/or can be ingested directly without scavenging all available saliva so as to cause an undesirable mouth feel, dry mouth and difficulty swallowing.

In embodiments in which the tablet compositions are suitable for reconstitution to an aqueous solution, a suitable volume of aqueous solution for reconstitution is about 25 ml, 50 ml, 75 ml, 100 ml, 125 ml, 150 ml, 175 ml, 200 ml, 225 ml or 250 ml. Ranges within one or more of the preceding values, e.g., 25-50 ml, 25-75 ml, 25-100 ml, 25-150 ml, 25-200 ml, 25-250 ml, 50-75 ml, 50-100 ml, 50-150 ml, 50-200 ml, 50-250 ml, 75-100 ml, 75-150 ml, 75-200 ml, 75-250 ml, 100-150 ml, 100-200 ml or 100-250 ml are contemplated by the invention. In particular embodiments, a suitable volume of solution for reconstitution is about 75 ml or about 100 ml.

The disclosed formulations produce dosage units that, when manufactured, exhibit consistent and acceptable physical characteristics. The dosage units must also be appropriate for ingestion by a patient and meet desirable and measurable pharmaceutical performance and quality attributes. Aspects of the compositions disclosed herein comprise sodium sulfate. In some embodiments, the sodium sulfate is selected from the group consisting of anhydrous sodium sulfate and sodium sulfate hydrates such as sodium sulfate decahydrate. The disclosed formulations further comprise potassium sulfate.

As used in certain aspects disclosed herein, formulations comprising sodium sulfate and potassium sulfate allow for sufficient laxative effects to induce laxation in subjects. Without being held to any particular theory, the sulfate salts are poorly absorbable and cause water to flow into the intestine when provided in the intestine in sufficient quantities. Poorly-absorbable salts exhibit limited uptake from the intestine and that the salts remaining in the intestine cause water to flow into the intestines. The formulations induce softening of stool to allow for spontaneous bowel movements (“SBM”) in subjects. Accordingly, the formulations disclosed herein can be administered to induce laxation (i.e., a bowel movement of loosened stool) of a subject (e.g., patient) and such compositions can be used to treat constipation induced by medications such as opiates and other medications.

A further advantage of the presently disclosed compositions is that such compositions do not cause clinically significant electrolyte shifts when administered in sufficient quantities to cause laxation and balanced with other salts as disclosed herein. The disclosed formulations avoid such shifts by providing sufficient amounts of sodium and potassium cations to avoid shifting the levels of these cations in a subject taking the compositions.

The formulations disclosed herein can be administered as solid oral dosage forms. Examples of solid oral dosage forms include a tablet, capsule, compressed capsule, gel capsule, dual purpose tablet, or caplet. Each solid oral dosage form can comprise from about 0.5 grams to about 1.5 grams of sodium sulfate and from about 0.2 grams to about 0.6 grams of potassium sulfate per solid oral dosage form. Each solid oral dosage form can also comprise from about 0.9 grams of sodium sulfate and about 0.4 grams of potassium sulfate per solid oral dosage form. In some embodiments, the formulation comprises about 0.6 grams to about 1.4 grams of sodium sulfate per solid oral dosage form or alternatively the formulation comprises from about 0.7 grams to about 1.3 grams of sodium sulfate per solid oral dosage form. The formulation can comprise from about 0.8 grams to about 1.2 grams out 0.8 grams to about 1.1 grams or from about 0.8 grams to about 1.0 grams of sodium sulfate per solid oral dosage form. The disclosed formulation can further comprise from about 0.3 grams of potassium sulfate to about 0.5 grams of potassium sulfate per solid oral dosage form. An exemplary formulation comprises 0.9233 grams of sodium sulfate per solid oral dosage form and 0.37833 of potassium sulfate per solid oral dosage form. In certain embodiments, each oral dosage form is a dose of the formulation.

It should be noted that the formulations can be administered on an as needed basis. In other words, a subject can administer the formulations disclosed herein when suffering from constipation until the constipation is resolved. In some embodiments, the subject administers the formulations disclosed herein daily. In other embodiments, the subject can administer the formulations in sufficient quantities to cause laxation. For instance, a subject can administer from 1 to 10 tablets to induce laxation. In certain instances, a subject will administer from 1 to 6 tablets to induce laxation. The formulations can be administered in doses from about 0.9 to about 8.0 grams or about 4.0 grams to about 8.0 grams of sodium sulfate and from about 0.3 grams to about 3.5 grams or 1.0 grams to about 3.5 grams of potassium sulfate. The formulations can be administered in doses from about 5.0 grams to about 7.0 grams of sodium sulfate and from about 2.0 grams to about 2.5 grams of potassium sulfate. In other embodiments, the formulations can be administered in doses of about 5.5 grams of sodium sulfate and about 2.7 grams of potassium sulfate. In more particular embodiments, the formulation can be administered in a dose of 5.54 grams of sodium sulfate and 2.27 grams of potassium sulfate.

In aspects of the solid oral dosage forms disclosed herein sodium sulfate and potassium sulfate can comprise at least 95% of the composition, at least 85% of the tablet composition, or at least 75% (w/w) of the composition. In some embodiments, the sodium sulfate and potassium sulfate comprise at least 60% (w/w) of the composition. In other embodiments, the sodium sulfate and the potassium sulfate comprise at least 50% (w/w) of the composition.

The solid oral dosage forms can further comprise one or more excipients. The one or more excipients (e.g., soluble) are selected from the group consisting of binders, lubricants, glidants, disintegrants, and combinations thereof. Exemplary excipients include binders such as copolyvidone, gelatin, hydroxypropyl cellulose, hydroxypropyl methylcellulose, hypromellose, lactose anhydrous, povidone, and polyethylene oxide. In certain embodiments, the one or more excipients include a disintegrant consisting of povidone, Kollidon CL, Kollidon CL-SF, sugar, sucrose, dextrose, mannitol, and a combination thereof; (ii) a combination of mannitol and povidone; and/or (iii) a combination of Kollidon CL and/or Kollidon CL-SF. Other exemplary excipients include emulsifying agents such as hydroxypropyl cellulose, polaxamer 407, and sodium lauryl sulfate. The formulations can comprise lubricants such as polyethylene glycol, polaxamer 407, sodium lauryl sulfate, sodium benzoate, sodium dodecyl sulfate, sodium caprylate, and sodium stearyl sulfate, magnesium stearate, stearic acid, hydrogenated vegetable oil, and glyceryl palmitostearate. Further exemplary excipients include disintegrants such as citric acid, croscarmellose sodium, and povidone.

In some embodiments, the solid oral dosage forms comprise only a very minimal amount of excipients. In certain embodiments, the solid oral dosage forms comprise less than or equal to 5% (w/w) excipients in the total tablet compositions. In other embodiments, the solid oral dosage forms comprise less than or equal to 10% (w/w) excipients and alternatively less than or equal to 5% (w/w) excipients in the total solid oral dosage forms.

It has also been discovered that commercially available sodium sulfate can form slow dissolving crystals. Such slow dissolving crystals can prevent a pharmaceutical tablet composition from dissolving sufficiently to have the desired effect on a subject. In certain embodiments, sodium sulfate particles having a diameter of less than 150 μm are removed prior to formulating pharmaceutical tablet compositions. In particular embodiments, the solid oral dosage form is substantially free of sodium sulfate particles of less than 150 μm. In some embodiments, fines of a diameter of less than 150 μm are added back to the sodium sulfate that is substantially free of such crystals. In particular embodiments, the fines comprise 10% (w/w) of the sodium sulfate. In particular embodiments, the sodium sulfate particles in the pharmaceutical tablet compositions comprises about 90% (w/w) particles having a diameter of between about 150 μm and about 700 μm and about 10% (w/w) fines (i.e., particles having a diameter of less than 150 μm) that are added back to the sodium sulfate. It is believed that the added fines increase hardness such that there is no picking. Furthermore, the formulations do not need additional lubricants beyond the levels disclosed herein. Additionally, the presently disclosed compositions allow for a wider hardness range for the compositions and a more robust formulation.

The solid oral dosage forms can be tableted using standard production style equipment and techniques (Bogda, Michael J. Ch. 260, “Tablet Compression: Machine Theory, Design, and Process Troubleshooting” in Encyclopedia of Pharmaceutical Technology, Third Edition, 2006). In other embodiments, the pharmaceutical tablet formulation is encapsulated.

Certain embodiments of the disclosed solid oral dosage forms further comprise a coating. For instance, the coating can be an enteric coating. Polymers useful for enteric coatings polymethacrylates such as methacrylic acid/ethyl acrylate, cellulose esters such as cellulose acetate phthalate (CAP), cellulose acetate trimellitate (CAT), hydroxypropylmethylcellulose acetate succinate (HPMCAS), and polyvinyl derivatives such as polyvinyl acetate phthalate (PVAP). In other embodiments, the coatings comprise hydroxypropylmethyl cellulose, methylcellulose, hydroxypropylcellulose, carboxymethylcellulose sodium, polyethylene glycols, synthetic polymers, shellac, corn protein zein, polysaccharides, polyvinyl alcohol, polyethylene glycol, Kollicoat IR (polyvinyl alcohol-polyethylene glycol co-polymer), Kollicoat SR 30D (Polyvinyl acetate stabilized with polyvinylpyrrolidone and sodium lauryl sulfate), or gelatin. Methods of making enteric coatings are well known in the art and include film formation and aqueous dispersion techniques (Porter S, Verseput R, Cunningham C. (1997) Pharm. Technol. 21: 60-70; Twitchell A, Hogan J, Aulton M. (1995a) S.T.P. Pharm. Sci. 5: 190-195; Twitchell A, Hogan J, Aulton M. (1995b) Drug Dev Ind Pharm 21:1611-1619; Tobiska S, Kleinbudde P. (2003) Pharm. Dev. Tech. 8: 39-46; Poukavoos N, Peck G. (1994) Drug Dev. Ind. Pharm. 20: 1535-1554; Wilson K, Crossman E. (1997) Drug Dev Ind Pharm 23:1239-1243).

The coating can also be a film that coats the tablet. Film coats are most commonly deposited on the tablets by spraying a thin uniform layer on the tablet. Polymers used in film coats include sucrose, hypromellose, hydroxyethyl cellulose, hydroxyethylmethyl cellulose, carboxymethylcellulose sodium, hydroxypropyl cellulose, polyethylene glycol, and ethylcellulose. In particular embodiments, the coating quickly dissolves without affecting disintegration and/or dissolution of the tablet composition. In certain embodiments, the tablet formulation comprises a coating that comprises Kollicoat IR and water.

Aspects of the pharmaceutical tablet compositions disintegrate rapidly in aqueous solutions such as gastrointestinal fluids. In some embodiments, the pharmaceutical tablet compositions disintegrate in less than 30 minutes. In particular embodiments, the pharmaceutical tablet compositions disintegrate in less than about 15 minutes. The disintegration time for such tablet compositions avoid ulcer complications.

The tablet formulation has the characteristic that at least 80% of the tablet formulation dissolves within 30 minutes using Apparatus 2. In certain embodiments, at least 75% of the tablet dissolves within 30 minutes using Apparatus 2. In other embodiments, at least 85% of the tablet formulation dissolves within 30 minutes using Apparatus 2. In particular embodiments, at least 90% of the tablet formulation dissolves within 30 minutes using Apparatus 2. In still more particular embodiments, at least 95% of the tablet formulation dissolves within 30 minutes using Apparatus 2. In other embodiments, at least 98% of the tablet formulation dissolves within 30 minutes using Apparatus 2. In some embodiments, at least 100% of the tablet formulation dissolves within 30 minutes using Apparatus 2.

The determination of tablet disintegration is accomplished using in vitro test methods provided in the United States Pharmacopoeia (see e.g., United States Pharmacopoeia, Vol. 36 <711> Dissolution, pages 307-313, applying Apparatus 2 (Paddle Apparatus)). Briefly, a paddle is formed from a blade and a shaft and is used as the stirring element driven by a motor at a specified speed (e.g., 50 rpm). The tablet is placed into an inert (typically glass) transparent vessel containing about 900 mL of the dissolving media (oftentimes water). The dissolution media in the vessel is maintained at 37° C. using a water bath, or other suitable means, to simulate human body temperature. The paddle is rotated in a uniform manner (minimal wobble) at a specified height above the bottom of the vessel, providing turbulence in the vessel similar to that of the human digestive tract. As the tablet dissolves, and after a specified period of time (e.g., 15 minutes, 30 minutes, or up to an hour), a sample of the dissolution media with the dissolved tablet is removed and tested for active ingredients. The test methods are specific for each active ion in the tablet (e.g., sodium, sulfate, potassium, and magnesium) and may utilize a specialized instrument (e.g., ion chromatograph, flame photometer), or some other testing means (e.g., titration). The test results may be set at, for example, Not Less Than (NLT) 80% (Q) in 15 minutes. The (Q), according to USP (refer to USP method <711> Interpretation for Immediate Release Dosage form, page 312), means that after testing 6 tablets (termed S1), the results of all 6 tablets are greater than Q+5, or 85%. If any tablets do not meet the criteria, another 6 tablets are tested (termed S2), and the average of the 12 tablets is equal to or greater than Q (80%), and no individual tablet result is less than Q-15 (65%). If any of the 12 tablets do not meet the criteria, another 12 tablets are tested (termed S3), and the average of the 24 tablets is equal to or greater than Q (80%), no more than 2 individual tablet results are less than Q-15 (65%), and no individual tablet result is less than Q-25 (55%). In particular embodiments, a dissolution media other than water (e.g. gastric fluid, buffered solution, etc.) is used. In particular embodiments, the paddle speed is adjusted to 75 rpm or higher. In still other embodiments, the sampling time is from about 30 minutes, about 45 minutes, or at least 60 minutes. In more embodiments, the Q is NLT 75% (Q), NLT 85% (Q), or any Q that is acceptable. In still other embodiments, a basket assembly is used.

In other aspects, the tablets are tested for physical disintegration by applying the USP Disintegration test (see USP Vol. 36 Physical Tests, method <701> Disintegration, pages 305-306 for plain coated or uncoated tablets). Six tablets are placed into separate tubes of a basket rack assembly of specified size and shape. The assembly is immersed in an alternating up and down motion into a beaker filled with disintegration media (e.g., water) maintained at 37° C. The apparatus may or may not have specialized disks for each tube. After a specified time period, the operator visually evaluates the apparatus for complete disintegration, defined as that state in which any residue of the unit (any of the 6 tablets), except fragments of insoluble coating or capsule shell, remaining on the screen of the test apparatus or adhering to the lower surface of the disk, if used, is a soft mass having no palpably firm core.

It should be noted that the formulations disclosed herein can be deviated from regarding the amounts of sodium sulfate and potassium sulfate so long as the laxative activity of the formulations is not affected. In particular, the formulations typically will have a ratio of sodium sulfate to potassium sulfate of at least about 2.5 to 1 and can have a ratio of about 3 to 1. In particular embodiments, the ratio of sodium sulfate to potassium sulfate is 2.44 to 1.

In certain embodiments, the Tablet formulation has the characteristics of a dual purpose (“DP”) solid dose tablet. In some embodiments the pharmaceutical tablet formulation compositions may be ingested as an immediate release tablet while optionally be quickly dissolved/disintegrated in an aqueous solution, such as water, for ingestion as a liquid.

The determination of DP Tablet disintegration is accomplished using a standard USP Disintegration test equipment. (see USP Vol. 36 Physical Tests, method <701> Disintegration, pages 305-306 for plain coated or uncoated tablets[use as a guide]). Six tablets are placed into separate tubes of a basket rack assembly of specified size and shape. The assembly is immersed in an alternating up and down motion into a beaker filled with disintegration media (e.g., water) maintained at or between 2° C., 8° C., 10° C., and 15° C. 85% of the tablets should be disintegrated in under 150 seconds. The apparatus may or may not have specialized disks for each tube. After a specified time period, the operator visually evaluates the apparatus for complete disintegration, defined as that state in which any residue of the unit (any of the 6 tablets), except fragments of insoluble coating or capsule shell or insoluble disintegrants, remaining on the screen of the test apparatus or adhering to the lower surface of the disk, if used, is a soft mass having no palpably firm core.

C. Powder Formulations

Other aspects disclosed herein include the disclosed formulations comprising a powder. Such formulations can comprise a dose of at least 6.0 grams of sulfate salts. In particular embodiments, the powder formulation comprises from about 4.0 grams of sodium sulfate to about 8.0 grams of sodium sulfate in combination with potassium sulfate in an amount from about 1.0 grams to about 3.5 grams in a total dose. In more particular embodiments, the powder formulation comprises about 5.5 grams of sodium sulfate and 2.7 grams of potassium sulfate in a total dose. In even more particular embodiments, the powder formulation comprises 5.54 grams of sodium sulfate and 2.27 grams of potassium sulfate in a total dose. Each dose can comprise from about 0.5 grams to about 1.5 grams of sodium sulfate and from about 0.2 grams to about 0.6 grams of potassium sulfate. Each dose can also comprise from about 0.9 grams of sodium sulfate and about 0.4 grams of potassium sulfate. In some embodiments, each dose comprises about 0.6 grams to about 1.4 grams of sodium sulfate or alternatively from about 0.7 grams to about 1.3 grams of sodium sulfate. Each dose can comprise from about 0.8 grams to about 1.2 grams out 0.8 grams to about 1.1 grams or from about 0.8 grams to about 1.0 grams of sodium sulfate. Each dose can further comprise from about 0.3 grams of potassium sulfate to about 0.5 grams of potassium sulfate. An exemplary dose comprises 0.9233 grams of sodium sulfate and 0.37833 of potassium sulfate.

When administered as a powder, the formulation can be consumed by reconstituting the powder into a sufficient volume of water to dissolve the sulfate salts and yet allow the sulfate salts to cause a laxative effect. Exemplary volumes of water include dissolving the sodium sulfate/potassium sulfate powder in 50 mL to 100 mL of water. In other embodiments, the powder formulation is dissolved in 50 mL to 200 mL of water. It should be noted that the volume of water can be adjusted so long as the laxative effective of the powder formulation is not adversely affected.

### Excipients

The pharmaceutical compositions of the present invention further include at least one excipient. As used herein, the term “excipient” refers to pharmacologically inactive substances that may be used, for example, to increase the volume or mass of a formulation, facilitate the manufacturing process, stabilize or protect active ingredients during storage, affect the solubility of the composition, affect the disintegration of the composition or improve palatability. Non-limiting examples of excipients for use in the present invention include anti-adherents, binders, coatings, disintegrants, fillers, flavors, colors, lubricants, glidants, sorbents, preservatives, sweeteners, and combinations thereof. In certain embodiments, the compositions of the invention do not include a preservative.

### II. Methods of Treatment

The present invention further provides methods to induce a bowel movement or to treat or prevent constipation in a subject by administering the oral pharmaceutical compositions disclosed herein to a subject, e.g., a subject suffering from drug-induced constipation, such as opioid-induced constipation.

The terms “treat” or “treating,” as used herein, refer to partially or completely alleviating, inhibiting, delaying onset of, reducing the incidence of, ameliorating and/or relieving constipation, or one or more symptoms of constipation, in one example, symptoms of opioid-induced constipation.

As used herein, the term “subject” refers to a mammal and includes human and animal subjects, such as domesticated animals (e.g., cows, horses, dogs, or cats) and experimental animals (e.g., mice, rats, dogs, chimpanzees, or apes). In a particular embodiment, the subject is human. In a particular embodiment, the subject is suffering from constipation, e.g., drug-induced constipation, such as opioid-induced constipation. In a particular embodiment, the subject is a cancer patient.

As used herein, the terms “suffer” or “suffering” refer to one or more conditions that a patient has been diagnosed with, or is suspected to have, in particular, constipation, e.g., drug-induced constipation, such as opioid-induced constipation.

As used herein, the term “constipation” refers to a condition in which a subject suffers from infrequent bowel movements or bowel movements that are painful and/or hard to pass. A subject experiencing constipation often suffers from straining during bowel movements and/or a sensation of incomplete evacuation following bowel movements. Specific clinical criteria for the diagnosis of constipation are provided by, for example, ROME III criteria (see, infra).

As used herein, the terms “bowel movement” or “laxation” or “laxative response” refer to the passage and evacuation of feces.

In one embodiment, the present invention provides methods to induce a bowel movement or to treat or prevent constipation in a subject by orally administering to a subject a composition of the invention. In one embodiment, the methods include direct oral ingestion of a composition of the invention by, e.g., by swallowing a composition of the invention as an aqueous solution, or by swallowing a composition of the invention as a tablet (e.g., 1, 2, 3, 4, 5, or 6 tablets). In another embodiment, the methods include oral ingestion of a composition of the invention by swallowing a composition of the invention as a reconstituted aqueous solution.

According to the methods described herein, the subjects are administered an effective amount of the compositions of the invention to induce a bowel movement. As used herein, an “effective amount” refers to the level required to induce laxation or to treat or prevent one or more symptoms of constipation. In some embodiments, an “effective amount” is at least a minimal amount of the compositions of the invention, which is sufficient for inducing a bowel movement or for treating or preventing constipation. In some embodiments, the term “effective amount,” as used in connection with an amount of sulfate ion or sulfate salt(s), refers to an amount of sulfate ion or sulfate salt(s), or compositions thereof sufficient to induce laxation or for treating or preventing at least one symptom of constipation.

To achieve the desired efficacious levels of laxation, the subject may be administered more than a single dose, depending on the composition being used. For example, the subject may be administered 1, 2 or 3 doses to induce a bowel movement. In various embodiments, the present invention includes methods for inducing a bowel movement in a subject, comprising orally administering to the subject the composition in a single dose for 1 or more days, for example for up to 28 consecutive days.

In some embodiment, to achieve the desired efficacious levels of laxation, the subject may be administered more than one tablet, depending on the composition being used. For example, the subject may be administered 1, 2, 3, 4, 5, 6 or more tablets to induce laxation.

Administration of the composition may induce 3 or more bowel movements within any 7 consecutive days of treatment. Administration of the composition may also induce at least one or more bowel movements from baseline within any 7 consecutive days of treatment. As used herein, the term “baseline” refers to the number of bowel movements experienced by the subject when the composition is not being administered. In a further embodiment, administration of the composition induces a response comprising 3 or more bowel movements within any 7 consecutive days of treatment and at least one or more bowel movement from baseline within any 7 consecutive days of treatment, during at least two, 7 consecutive day treatment periods. As used herein, a “treatment period” refers to a period of hours, days, weeks or months when the composition is administered to a subject. In a particular embodiment, the composition induces the response during at least three, 7 day consecutive day treatment periods.

In various embodiments, the present invention includes methods for inducing a bowel movement in a subject, comprising orally administering to the subject a composition of the invention such that the subject no longer meets a ROME III criteria at the end of a 7 day treatment period, the subject experiences a bowel movement within 3 hours of the first dose and/or the subject experiences a bowel movement within 3 hours of the first dose. As used herein, ROME III provides clinical criteria for the diagnosis of constipation. According to the ROME III criteria, a subject is constipated if the subject reports 2 or more of the following symptoms 1) straining during at least 25% of defecations, 2) lumpy or hard stools in at least 25% of defecations, 3) sensation of incomplete evacuation for at least 25% of defecations, 4) sensation of anorectal obstruction/blockage for at least 25% of defecations, 5) manual maneuvers to facilitate at 25% of defecations or 6) fewer than 3 defecations per week (Longstreth et al., Gastroenterology (2006) 130(5):1480-1491).

The methods of the invention also include a method for inducing a bowel movement in a subject, comprising orally administering to the subject a composition of the invention and allowing the composition to induce a bowel movement in the subject. Another aspect of the invention provides methods for treating or preventing constipation in a subject by orally administering to a subject the composition of the invention and allowing the composition to induce a bowel movement in the subject. According to the method, one or more bowel movements may be produced in 1 to 24 hours following administration of the composition. For example, the bowel movement may be produced in 1, 2, 3, 4 or 5 hours following administration of the composition. In a particular embodiment, the method may induce a bowel movement within 5 hours of administration. In another embodiment, the method may induce a bowel movement within 4 hours of administration. In another embodiment, the method may induce a bowel movement within 3 or 2 hours of administration.

Treatment of the subject with a composition of the invention may also induce 1 to 6 bowel movements within 24 hours of administration. For example, the composition may induce 2, 3, 4 or 5 bowel movements within 24 hours administration. Treatment of the subject with the composition may also induce one or more bowel movements within 24 hours of administration. In yet another embodiment, following administration of the composition to the subject for 7 consecutive days, the subject no longer meets the ROME III criteria for constipation.

The pharmaceutical compositions of the invention may be taken as necessary by the subject. For example, the pharmaceutical compositions of the invention may be taken on demand to induce an immediate and desired bowel movement. Alternatively, the pharmaceutical compositions may be taken on a regular dosing regimen, for example, once a day or twice a day. Such regimens may be for at least 1 week, 2 weeks, 3 weeks, 4 weeks or more.

According to the invention, administration of the oral compositions of the invention, induces a bowel movement to treat or prevent constipation. In one embodiment of the invention, the constipation is drug induced-constipation. As used herein, the term “drug-induced constipation” refers to a condition in which a subject suffers from infrequent bowel movements or bowel movements that are painful and/or hard to pass caused by using a drug or a combination of drugs. A subject experiencing drug-induced constipation often suffers from straining during bowel movements and/or a sensation of incomplete evacuation following bowel movements.

A “drug that induces drug-induced constipation” is one that, for example, induces a 10% or higher incidence of constipation in a subject administered the drug as reported on the FDA label of the drug, e.g., reported as an adverse event associated with the drug.

Drugs which may cause drug-induced constipation include a variety of different medications such as opioids, anticholinergic agents, tricyclic antidepressants (e.g., amytriptyline, nortriptyline, amoxapine, trimipramine, aminotriptyline, imipramine, clomipramine, dosulepin, lofepamine), tetracyclic antidepressants (e.g., setiptiline, maprotiline, mianserin), calcium channel blockers (e.g., verapamil), antiparkinsonian drugs (e.g., trihexyphenidyl, levodopa), sympathomimetics (e.g., ephedrine, terbutaline), antipsychotics (e.g., chlorpromazine), diuretics (e.g., furosemide), anti-incontinence agents (e.g., propanetheline, oxybutynin), benzodiazepine drugs, phenothiazine drugs (e.g., chlorpromazine) antihistmines (e.g., diphenyldramine), antacids (e.g., aluminum preparations), calcium supplements, iron, antidiarrheal agents (e.g., loperamide, attapulgite), iron preparations, and nonsteroidal anti-inflammatory drugs (e.g., ibuprofen). Further examples of drugs which may cause drug-induced constipation are provided in the Table below.

| Brand Name | Generic Name | Type/class of Drug |
|---|---|---|
| Hycet, Lortab Elixir, | acetaminophen and | opioid |
| Norco, Vicodin, Xodol, | hydrocodone | |
| Zamicet, Zolvit | | |
| Binosto, Fosamax | alendronate | bisphosphonates |
| Niravam, Xanax, Xanax | alprazolam | benzodiazepines |
| XR | | |
| Symmetrel, Endantadine | amantadine hydrochloride | adamantane antivirals, |
| | | dopaminergic |
| | | antiparkinsonism agents |
| Cordarone, Pacerone, | amiodarone | Group III antiarrhythmics |
| Nexterone | | |
| Vanatrip, Levate. Elavil | amitriptyline | tricyclic antidepressants |
| Asendin | amoxapine | tricyclic antidepressants |
| Soma Compound | aspirin and carisoprodol | skeletal muscle relaxant |
| | | combinations |
| Darvon Compound 32, | aspirin/caffeine/propoxyphene | narcotic analgesics |
| Darvon Compound-65, | | |
| PC-CAP, Propoxyphene | | |
| Compound 65 | | |
| Cycloset, Parlodel | bromocriptine | dopaminergic |
| | | antiparkinsonism agents, |
| | | prolactin inhibitors |
| Aplenzin, Buproban, | bupropion | miscellaneous |
| Wellbutrin, Wellbutrin | | antidepressant, smoking |
| SR, Wellburtin XL, | | cessation agents |
| Zyban, Zyban Advantage, | | |
| Budeprion, Budeprion SR, | | |
| Budeprion XL, Forfivo, | | |
| Forfivo XL | | |
| Evadene, Evadyne, | butriptyline | tricyclic antidepressants |
| Evasidol, Centrolese | | |
| Lodosyn | carbidopa | dopaminergic |
| | | antiparkinsonism agents |
| Atamet, Parcopa, Sinemet, | carbidopa/levadopa | dopaminergic |
| Sinemet CR | | antiparkinsonism agents |
| Stalevo | carbidopa/levodopa/entacapone | dopaminergic |
| | | antiparkinsonism agents |
| Cholestyramine Light, | cholestyramine | bile acid sequestrants |
| Prevalite, Prevalite | | |
| Packets, Questran, | | |
| Questran Light, Questran | | |
| Light Packets, Questran | | |
| Packets, LoCholest | | |
| Anafranil | clomipramine | tricyclic antidepressants |
| Catapres, Kapvay, Kapvay | clonidine | antiadrenergic agents, |
| Dose Pack, Jenloga, | | centrally acting |
| Nexiclon XR, Kapvay, | | |
| Duraclon | | |
| Welchol | colesevelam | bile acid sequestrants |
| Enablex | darifenacin | urinary antispasmodics |
| Norpramin | desipramine | tricyclic antidepressants |
| Depakote, Depakote ER, | divalproex sodium | fatty acid derivative |
| Depakote Sprinkles | | anticonvulsants |
| Silenor, Prudoxin | doxepin | miscellaneous anxiolytics, |
| | | sedatives and hypnotics, |
| | | tricyclic antidepressants |
| Comtan | entacapone | dopaminergic |
| | | antiparkinsonism agents |
| Epogen, Procrit, Eprex ® | epoetin alfa | recombinant human |
| | | erythropoietins |
| Ultradol ™, Lodine, | etodolac | nonsteroidal anti- |
| Lodine XL, Eccoxolac. | | inflammatory agents |
| Ebretin | | |
| Felbatol | felbamate | carbamate anticonvulsants |
| Zohydro ER | hydrocodon | oioid |
| Ibudone, Reprexain, | hydrocodone/ibuprofen | opioid |
| Vicoprofen | | |
| Dilaudid ®, Exalgo, | hydromorphone hydrochloride | opioid |
| Hydrostat IR | | |
| Tofranil, Tofranil-PM | imipramine or melipramine | tricyclic antidepressants |
| Roferon-A | interferon alfa-2a, recombinant | antineoplastic interferons |
| Intron A | interferon alfa-2b | antineoplastic interferons |
| Infergen | interferon alfacon-1 | interferons |
| Alferon N | interferon alfa-n3 | interferons |
| | iron supplements | |
| Madopar, Prolopa HBS | levodopa | dopaminergic |
| | | antiparkinsonism agents |
| Orlaam | levomethadyl | opioid |
| Levo-Dromoran | levorphanol | narcotic analgesics |
| Altoprev, Mevacor | lovastatin | statins (HMG-CoA |
| | | reductase inhibitors) |
| Pethidine, Demerol HCl | meperidine | opioid |
| Mepergan | meperidine hydrochloride and | narcotic analgesics |
| | promethazine hydrochloride | |
| Dolophine, Methadose, | methadone | opioid |
| Methadose Sugar-Free | | |
| Astramorph/PF ™, | morphine | opioid |
| Duramorph ®, Avinza ®, | | |
| Kadian ®, MS Contin ®, | | |
| DepoDur ™, Infumorph ®, | | |
| Oramorph SR ®, | | |
| Roxanol ™ | | |
| DepoDur | morphine sulfate extended- | opioid |
| | release liposome injection | |
| Adalat CC, Afeditab CR, | nifedipine | calcium channel blocking |
| Nifediac CC, Nifedical | | agents |
| XL, Procardia, Procardia | | |
| XL | | |
| Aventyl and Pamelor | nortriptyline | tricyclic antidepressants |
| Zofran ®, Zofran ODT ®, | ondansetron | antiemetic |
| Zuplenz | | |
| Ditropan XL, Urotro | oxybutynin | urinary antispasmodics |
| OxylR, Oxecta, | oxycodone | opioid |
| OxyContin, Oxyfast, | | |
| Roxicodone | | |
| Opana, Opana ER, | oxymorphone | opioid |
| Numorphan | | |
| Aredia | pamidronate | bisphosphonates |
| Brisdelle, Paxil, Paxil CR, | paroxetine | selective serotonin reuptake |
| Pexeva | | inhibitors (SSRIs) |
| Dilantin, Dilantin | phenytoin | Group I antiarrhythmics, |
| Infatabs, Dilantin-125, | | hydantoin anticonvulsants |
| Phenytek, Dilantin | | |
| Kapseals, Phenytoin | | |
| Sodium, Prompt, Di-Phen | | |
| Phenadoz, Phenergan | promethazine | antihistamines, |
| | | phenothiazine antiemetics |
| Darvon, Darvon-N, | propoxyphene | |
| Dolene | | |
| Vivactil | protriptyline | tricyclic antidepressants |
| Actonel | risedronate | bisphosphonates |
| Actonel with Calcium | risedronate and calcium | bisphosphonates |
| | carbonate | |
| Zoloft | sertraline | selective serotonin reuptake |
| | | inhibitors (SSRIs) |
| VESIcare | solifenacin | urinary antispasmodics |
| Carafate | sucralfate | miscellaneous GI agents |
| Nucynta, Nucynta ER, | tapentadol | opioid |
| Palexia, Tapal | | |
| | tofranil | |
| Tasmar | tolcapone | dopaminergic |
| | | antiparkinsonism agents |
| Detrol, Detrol LA | tolterodine | urinary antispasmodics |
| Ultram, Ultram ER, | tramadol, tramadol | opioid |
| Conzip, Rybix ®, Rybix | hydrochloride | |
| ODT, Ryzolt | | |
| Surmontil | trimipramine | tricyclic antidepressants |
| Actigall, BILIVER, Coric, | ursodiol or usodeoxycholic | gallstone solubilizing agents |
| Deursil, Egyurso, Udiliv, | acid | |
| UDOXYL, Urso, Urso | | |
| Forte, Ursocol, | | |
| Ursofalk, Urso DS and | | |
| Ursosan | | |
| Depacon | valproate | fatty acid derivative |
| | | anticonvulsants |
| Effexor, Effexor XR | venlafaxine | S/NRI-antidepressant |
| Calan, Calan SR, Isoptin | verapamil | calcium channel blocking |
| SR, Verelan, Verelan | | agents, Group IV |
| PM, Isoptin, Isoptin I.V., | | antiarrhythmics |
| Covera-HS ® | | |
| Reclast, Zometa | zoledronic acid | bisphosphonates |

In a particular embodiment, the drug-induced constipation is “opioid-induced constipation.” In one embodiment, the opioid is selected from the group consisting of alfentanil, anileridine, asimodiline, bremazocine, burprenorphine, butorphanol, codeine, dezocine, diacetylmorphine, dihydrocodein, diphenyloxylate, fedotozine, fentanyl, funaltrexamine, hydrocodone, hydromorphone, levallorphan, levomethadyl acetate, levorphanol, loperamide, meperidine, methadone, morphine, morphine-6-glucoronide, nalbupine, nalorphine, opium, oxycodone, oxymorphone, pentazocine, propiram, propoxyphene, remifentanyl, sufentanil, tilidine, trimebutine, and tramadol.

In one embodiment of the invention, the subject is receiving opioids acutely. In another embodiment, the subject is receiving opioids chronically. In another embodiment, the subject is a cancer patient receiving opioids acutely or chronically.

According to the invention, administration of the oral compositions of the invention induces a bowel movement to treat or prevent constipation without producing a clinically significant electrolyte shift, e.g., a clinically significant sodium and potassium shift, in the subject. The term “clinically significant” is well known and understood in the art. Clinically significant electrolyte imbalances or shifts can be caused by undesirable electrolyte secretion. Accordingly, in a particular embodiment, the oral sulfate salt compositions of the invention induce laxation and/or treat or prevent constipation without a clinically significant alternation of, for example, sodium, chloride, bicarbonate, potassium, calcium, and/or phosphate level and balance in the subject to whom the composition is administered.

The effect of the compositions of the invention on electrolyte balance can be assessed by, for example, the methods described in U.S. Pat. No. 6,946,149 (Cleveland), issued Sep. 20, 2005, the entire contents of which are hereby incorporated by reference herein.

This invention is further illustrated by the following examples which should not be construed as limiting. The contents of all references, patents and published patent applications cited throughout this application, as well as the figures, are incorporated herein by reference.

### EXAMPLES

### Example 1: The Development of a Low Dose Sulfate Solution as a Laxative for Constipation in Normal Volunteers

### Summary

This single center study evaluated sulfate salt compositions containing 43.8 millimoles (mmol) sulfate salts (4.2 g total sulfate) or 80 mmol sulfate salts (7.7 g total sulfate) as compared to 10 mg bisacodyl for efficacy to induce a rapid, controlled bowel movement without significant gains or losses of electrolytes. Normal volunteers were given a single dose of laxative in an in-patient setting during which all stool and urine were collected for 24 hours after laxative administration. Efficacy was based on total stool output, bowel movement frequency and time to first bowel movement (BM). A time to first BM of less than three hours was considered ideal. Stool was analyzed to determine the gain or loss of electrolytes. Safety was assessed through the collection of adverse event data and analysis of urine electrolyte composition.

A sulfate formula containing 43.8 mmol sulfate salts induced less stool and fewer bowel movements over a 12 hour period than 10 mg bisacodyl. Sulfate formulas containing 80 mmol sulfate salts induced about the same amount of stool and bowel movements as 10 mg bisacodyl. The 80 mmol sulfate salt formulation, but not the 43.8 mmol sulfate salt formulation, had a much faster time of onset inducing a bowel movement in less than three hours in all subjects. Only one bisacodyl volunteer had a bowel movement in less than three hours. No study volunteer reported incontinence in association with any of the test laxatives.

Including stool output data from a previous study by the same investigators for a 155 mmol sulfate salt dose, a statistically significant dose response was demonstrated.

A formulation providing a 3:1 ratio of sodium to potassium minimized sodium and potassium electrolyte absorption or loss.

Gastrointestinal symptoms were similar between the 10 mg bisacodyl and the sulfate formulations, although one bisacodyl volunteer reported severe cramping. There were no on-study deaths.

### Study Objective

The purpose of the current study was to determine an approximate dose and composition for a sulfate based laxative in normal volunteers.

### Study Plan

Overall Study Design and Plan

Normal male and female volunteers in this single center study were enrolled to receive a single dose of a sulfate laxative formulations or bisacodyl in an open-label, non-randomized fashion.

Study Design: Including Choice of Control Groups

Bisacodyl was selected as the active control. This is a widely used OTC chemical stimulant laxative.

Selection of Study Population

A. Inclusion Criteria:

Male or female subjects that met the following criteria were eligible for enrollment:
1) Subjects in good health, as judged by a physical examination and review of medical history.
2) Subject must be 18 to 60 years of age.
3) If female and of childbearing age, is using an acceptable form of birth control.
4) If female, a negative birth control test, if applicable
5) In the investigator's judgment, subject must be mentally competent to sign an instrument of informed consent.

B. Exclusion Criteria:

Subjects who met any of the following criteria were excluded from the study:
1) Subjects with renal failure (creatinine >1.5 mg/dL), liver disease, congestive heart failure, or history of renal stones.
2) Subjects who are breastfeeding, pregnant or intend to become pregnant during the study.
3) Subject is of childbearing potential and refuses a pregnancy test.
4) Subjects allergic to bisacodyl or sulfate.
5) Subjects who in the opinion of the investigator should not be included in the study for any reason including inability to follow study procedures.

Treatments

A. Treatments Administered:

Enrolled study subjects received a single dose of a formulation of sulfate salts (dissolved in 120 ml water) and separately, 10 mg bisacodyl, in an open-label, non-randomized fashion. The sulfate preparations, shown below, were compounded by the investigator.

Sulfate Formulation (43.8 mmol sulfate salts):in 120 ml water

| | |
|---|---|
| Mallinckrodt Baker, Inc., Paris, KY | 3.11 g Sodium sulfate |
| Mallinckrodt Baker, Inc., Paris, KY | 3.82 g Potassium sulfate |

1:1 Sulfate Formulation (80 mmol sulfate salts):in 120 ml water

| | |
|---|---|
| Mallinckrodt Baker, Inc., | 5.68 g Sodium sulfate |
| Paris, KY | |
| Mallinckrodt Baker, Inc., | 6.97 g Potassium sulfate |
| Paris, KY | |

3:1 Sulfate Formulation (80 mmol sulfate salts):in 120 ml water

| | |
|---|---|
| Mallinckrodt Baker, Inc., | 8.52 g Sodium sulfate |
| Paris, KY | |
| Mallinckrodt Baker, Inc., | 3.49 g Potassium sulfate |
| Paris, KY | |

Bisacodyl (Dulcolax, 5 mg tablets): Boeringer Ingleheim

B. Method of Assigning Subjects to Treatment Groups:

This was an open-label study.

C. Selection of Dose in the Study:

One purpose of the study was to identify a starting dose for later clinical study. The sulfate formulation dose was compared to the effect of a dose of 10 mg bisacodyl.

D. Selection and Timing of Dose for Each Subject:

After an eight hour (overnight) fast, study subjects ingested either a single dose of sulfate laxative (containing sodium sulfate and potassium sulfate) or, on a different day, bisacodyl. Sulfate was initially given with a 1:1 molar ratio sodium to potassium at a dose of 43.8 mmole sulfate salts (4.2 g total sulfate) sulfate to three study subjects. The dose was later increased to 80 mmole sulfate salts (7.7 g total sulfate) in one study subject. The 80 mmol sulfate salt formulation was then changed to a 3:1 sodium to potassium molar ratio for the final five study subjects. Bisacodyl was given at a dose of 10 mg.

Stool and urine were collected for 24 hours following laxative ingestion.

E. Blinding:

This was an open-label study.

F. Prior and Concomitant Therapy:

Subjects taking medications for significant medical issues were ineligible for study enrollment. Concomitant medications were not allowed during the study period.

G. Treatment Compliance:

Study preparations were administered at the study center under close supervision. Study personnel ensured that all subjects were 100% compliant with preparation administration.

H. Prior and Concomitant Therapy:

Subjects taking medications for significant medical issues were ineligible for study enrollment. Concomitant medications were not allowed during the study period.

I. Treatment Compliance:

Study preparations were administered at the study center under close supervision. Study personnel ensured that all subjects were 100% compliant with preparation administration.

Efficacy and Safety Variables

A. Efficacy Measurements Assessed

Efficacy was assessed via quantitative measurements of stool weight, bowel movement frequency and time to first BM. All stools were collected, weighed and subsequently analyzed for water and electrolyte composition.

B. Safety Measurements Assessed

Laxative related adverse events were assessed via subject completion of a study specific questionnaire. Specifically, subjects rated their experience with respect to abdominal cramping, abdominal bloating, abdominal pain, gas, nausea, and vomiting. Stool was analyzed for water and electrolyte composition.

Statistical Methods

A. Statistical and Analytical Plans:

Where performed, comparisons between treatments were calculated by t-test. A probability result of 0.05% was considered statistically significant.

B. Determination of Sample Size:

This study was designed to enroll five to ten subjects. The study sample size is consistent with previous studies using this dosing technique and stool collection methodology.

### Study Subjects

Disposition of Subjects

Six male and 1 female normal volunteers were enrolled into the study. Treatment assignments, with the corresponding study numbers, are shown in the table below:

TABLE 1
Volunteer Treatment Assignment

| | 10 mg | 43.8 mmol | 80 mmol |
|---|---|---|---|
| Volunteer | bisacodyl | Sulfate salt | Sulfate salt |
| DC (A) | 3266 | — | 3253* |
| RH (B) | 3262 | — | 3255 |
| BW (C) | 3252 | 3249 | 3256 |
| BT (D) | 3269 | — | 3263 |
| Cku (E) | 3264 | — | 3268 |
| CK (F) | 3247 | 3250 | 3267 |
| ZB | 3251 | 3248 | — |

*1:1 Na:K formula

The letter identifier (A-F) indicates volunteers that took both the 10 mg bisacodyl and 80 mmol sulfate salt doses.

Protocol Deviations

No significant protocol deviations were reported.

### Efficacy Evaluations

Data Sets Analyzed

Seven study subjects were enrolled in this open label study.

Demographic and Other Baseline Characteristics

The study population demographics are summarized in Table 2 below. The study included six male and one female normal volunteers.

TABLE 2
Study Demographics

| Age (years) | |
|---|---|
| n | 7 |
| Mean (SD) | 31.3 (7.5) |
| Range | 25-45 |
| Race | |
| Caucasian | 4 |
| African-Amer. | 2 |
| Asian | 1 |

SD = standard deviation

Measurements of Treatment Compliance

Study subjects were administered study medication in an in-patient setting. All subjects were 100% compliant with preparation administration.

Efficacy Results and Tabulations

A. Efficacy Measures:

Study efficacy endpoints were the time to first bowel movement, bowel movement frequency and total stool output over a 24 hour period after administration of the study laxative. These are shown below for the two sulfate salt doses and bisacodyl.

TABLE 3
Bowel Movement Measures

| | Bisacodyl | | Sulfate salt | | Sulfate salt | |
|---|---|---|---|---|---|---|
| Measure (SD) | 10 mg | | 43.8 mmol | | 80 mmol | |
| n | 7 | | 3 | | 6 | |
| Stool Output (g/12 hrs) | 681 | (232) | 288 | (131) | 802 | (187) |
| BM Frequency (#/12 hrs) | 2.9 | (1.2) | 1.7 | (1.2) | 4.3 | (1.9) |
| Time to first BM (hrs) | 4.4 | (1.8) | 5.8 | (3.8) | 1.6 | (0.8) |
| Subjects with BM <3 hrs | 1 | (14%) | 1 | (33%) | 6 | (100%) |

Table 3 shows that subjects treated with a sulfate formula containing 80 mmol sulfate salts (7.7 grams total sulfate) averaged about 4 bowel movements and 800 grams of stool over the 24 hour study period. The first bowel movement generally occurred within 2 hours from the time the laxative dose was ingested. All study subjects taking the 80 mmol sulfate salt formula had three or more BM's and no study subject had their first bowel movement later than 3 hours following laxative ingestion. Study subjects taking 10 mg bisacodyl averaged about 3 BM and nearly 700 grams of stool for the 24 hours following laxative ingestion, however, the bisacodyl was comparatively slow to act taking about 5 hours for the first bowel movement to occur. Study subjects that took the 43.8 mmol sulfate salt (4.2 grams total sulfate) dose had much lower stool output. All study subjects that took 10 mg bisacodyl or the 43.8 mmol sulfate salt dose had at least 1 BM during the study period and only one study subject in both the 43.8 mmol sulfate salt dose and 10 mg bisacodyl group had their first BM in less than 3 hours after laxative ingestion.

No study volunteer reported incontinence in association with any of the test laxatives.

Previous studies which followed similar protocols provide additional useful information. In these studies, 20 mg bisacodyl (Study 005-082) or a 155 mmol sulfate salt (14.9 g total sulfate) dose (see, e.g., Study 006-181 and see Patel et al., Amer J Gastroenterol (2009) 104(4):953-965) were given to normal volunteers. In study 006-181, which was intended to induce diarrhea for colon cleansing, a sulfate formulation containing three sulfate salts (sodium sulfate, potassium sulfate and magnesium sulfate) was used in 5 volunteers. The results of these studies for 20 mg bisacodyl and 155 mmol sulfate are shown in Table 4, below.

TABLE 4
Bowel Movement Measures
Previous Studies

| | Bisacodyl | | Sulfate | |
|---|---|---|---|---|
| Measure (SD) | 20 mg | | 155 mmol | |
| Study | 005-082 | | 006-181 | |
| n | 11 | | 5 | |
| Stool Output (g/12hrs) | 757 | (260) | 1305 | (590) |
| BM Frequency (#/12hrs) | 5.4 | (2.2) | 4.2 | (1.1) |
| Time to first BM (hrs) | 3.2 | (1.4) | 1.6 | (1.0) |
| # with BM <3 hrs | 5 | (45%) | 4 | (80%) |

As shown above in Tables 3 and 4, 20 mg bisacodyl did not substantially increase stool output compared to 10 mg bisacodyl, and only modestly improved the time to the first bowel movement. The number of study subjects with a bowel movement in less than 3 hours increased to only 45% (from 14% with 10 mg bisacodyl; Table 3).

Table 4 also shows that that the 155 mmol sulfate salt dose greatly increased total stool output over the 80 mmol sulfate salt dose in Table 3, but there was no difference in the total number of bowel movements. These stools tended to be liquid. Similar to the 80 mmol sulfate salt dose shown in Table 3, most (4 of 5) of study subjects taking the 155 mmol sulfate salt dose produced a BM within 3 hours.

B. Stool Water:

Stool collected from study subjects was analyzed for water content. This is shown below in Table 5 for 10 mg bisacodyl and the 43.8 mmol and 80 mmol sulfate salt doses. In addition, stool water content from the 155 mmol sulfate salt dose from the 5 study subjects in the previous 006-181 study is also presented.

TABLE 5
Stool Water Content

| | Bisacodyl | Sulfate salt | Sulfate salt | Sulfate |
|---|---|---|---|---|
| Measure (SD) | 10 mg | 43.8 mmol | 80 mmol | 155 mmol* |
| n | 7 | 3 | 6 | 5 |
| Stool Water (%) | 85.8% (2.4) | 81.3% (10.2) | 92.9% (1.8) | 94.1% (0.8) |

*Data from Study 006-181

As indicated by Table 5, there was little difference between the 80 mmol sulfate salt and 155 mmol sulfate salt doses with respect to stool water content. Both doses tended to produce very soft or liquid stools unlike the 43.8 mmol sulfate salt dose.

C. Stool Electrolytes:

Stools collected from study subjects were analyzed for sodium, potassium and sulfate. The data for the 80 mmol sulfate salt formulation and 10 mg bisacodyl are discussed here. The total electrolyte content of the stool was compared with the total electrolyte quantity ingested from the test preparations.

The 80 mmol sulfate salt formulation was administered initially as a formula with a 1:1 sodium to potassium ratio on a molar basis (1 study subject; DC). Based on the stool analysis results from this subject (see below) the formulation was modified to a 3:1 sodium to potassium ratio for the remaining 5 study subjects. Stool electrolyte balance results are summarized below in Tables 6 and 7.

TABLE 6
Mean Stool Sodium Output and Balance (mM)

| | | | 1:1 Sulfate | 3:1 Sulfate | |
|---|---|---|---|---|---|
| | Bisacodyl | | salt | salt | |
| Measure (SD) | 10 mg | | 80 mmol | 80 mmol | |
| n | 7 | | 1 | 5 | |
| Na Input | 0 | | 80 | 120 | |
| Na Output | 53.4 | (25.7) | 92.1 | 126.1 | (31.4) |
| +Gain/−Loss | −53.4 | (25.7) | −12.1 | −6.1 | (31.4) |

Table 6 shows that the sulfate formulations resulted in no or minimal sodium movement. The bisacodyl induced much larger sodium losses (1.2 grams).

TABLE 7
Mean Stool Potassium Output (mM)

| | | | 1:1 Sulfate | 3:1 Sulfate | |
|---|---|---|---|---|---|
| | Bisacodyl | | salt | salt | |
| Measure (SD) | 10 mg | | 80 mmol | 80 mmol | |
| n | 7 | | 1 | 5 | |
| K Input | 0 | | 80 | 40 | |
| K Output | 46.8 | (18.5) | 27.8 | 31.9 | (7.5) |
| +Gain/−Loss | −46.8 | (18.5) | 52.2 | 8.1 | (7.5) |

Table 7 shows that the 3:1 sulfate salt formulation resulted in no or minimal potassium gain or loss. The 1:1 sulfate salt formula caused a substantial potassium gain (about 2 grams) and bisacodyl a loss (also about 2 grams).

Study subjects taking the 80 mmol sulfate salt laxative gained about 21 mmol of sulfate (about 2 grams).

D. Drug Dose Response:

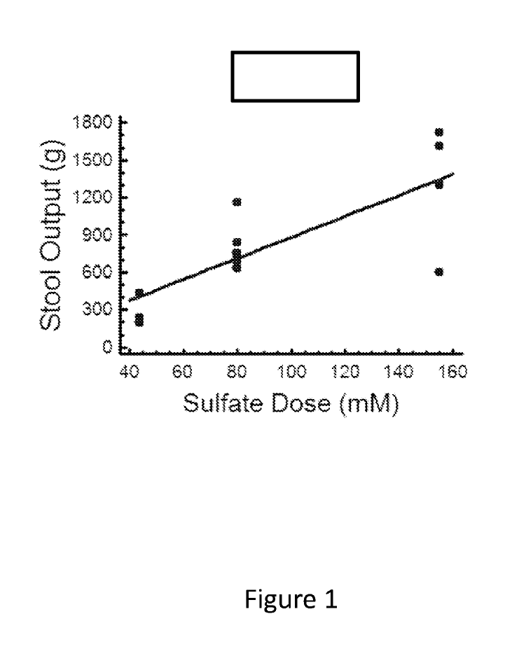

Combining stool output data for the 43.8 mmol and 80 mmol sulfate salt formulations in this study with the stool output data from the 155 mM formulation used in Study 006-181, a linear dose response of stool weight in response to sulfate dose is clearly demonstrated as shown in FIG. 1.

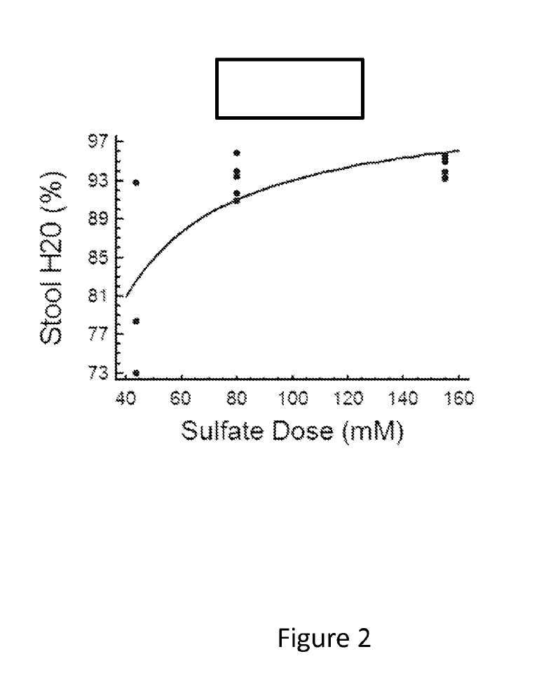

The dose response data analyzed by linear regression yields a correlation coefficient of 0.80 (F=21.46; p<0.001). The sulfate water content data can be similarly analyzed and is shown in FIG. 2.

For the analysis of water content, the fitted line has a correlation coefficient of −0.744 (F=14.9; p=0.0023). FIG. 2 shows that there is little difference in water content between the 80 mmol sulfate salt and 155 mmol sulfate salt doses, and both tend to be very soft or liquid.

A sulfate dose of 80 mmol sulfate salt (7.7 grams total sulfate) induces a bowel movement well within the desired 3 hour time frame with minimal side effects in normal volunteers (see below). This is about half the time required for 10 mg bisacodyl or a sulfate dose of 43.8 mmol sulfate salt (4.2 grams total sulfate). Similarly, the number of bowel movements is about twice that caused by 10 mg bisacodyl although the total stool output is nearly equivalent. A sulfate dose of 155 mmol sulfate salt (14.9 grams total sulfate) increases stool output over the 80 mmol sulfate salt dose by about 60% without substantially increasing the number of bowel movements or stool water content.

Ideally, a dose that provides a BM within three hours of laxative ingestion in most subjects, but with fewer total, non-liquid, bowel movements and less stool output (as well as lower water content) than that observed for the 80 mmol sulfate salt dose would be optimal. Therefore, a comfortable therapeutic dose for a sulfate laxative for constipated subjects is expected to be between 43.8 mmol and 80 mmol sulfate salt, represented by the curved portion of the line on FIG. 2. A reasonable starting dose for further study is about 60 mmol sulfate salt (5.8 g total sulfate).

E. Efficacy Conclusions:

A single dose of 80 mmol sulfate salt is highly effective in producing a bowel movement within three hours or less. The 3:1 ratio of sodium sulfate to potassium sulfate minimizes sodium and potassium movement. Bisacodyl, typical of laxatives, induces large potassium losses and is slow to act.

Including stool output data for a 155 mmol sulfate salt dose from a previous study of similar design by the same investigators, a statistically significant dose response was demonstrated.

### Safety Evaluation

The sulfate formulations were associated with very mild gastrointestinal symptoms. There were no unusual changes in urine electrolytes.

There were no on-study deaths. There was one unexpected adverse event of leg cramps in a 3:1 sulfate formula study subject.

### Conclusion

This study evaluated oral sulfate laxative formulations containing 43.8 mmol sulfate salt or 80 mmol sulfate salt to 10 mg bisacodyl in normal volunteers. The 80 mmol sulfate salt formula induced about the same amount of stool and bowel movements as 10 mg bisacodyl, although the sulfate formulation had a much faster time of onset inducing a bowel movement in less than three hours in all study subjects.

A linear dose response of stool output to sulfate dose was demonstrated.

A formulation providing a sodium sulfate to potassium sulfate ratio of 3:1 minimized sodium and potassium electrolyte absorption or loss.

Gastrointestinal symptoms were similar between the 10 mg bisacodyl and the sulfate formulation.

### Example 2: A Pilot Efficacy Evaluation of BLI 801 Laxative in Constipated Adults

### Summary

This multi-center study evaluated a seven day treatment of BLI801 Laxative in adult outpatients meeting ROME III constipation criteria. The intent of the study was to determine the efficacy of the laxative as both a one day and a seven day therapy.

Fifty-two patients were randomized, 43 female and 9 male. Thirty-three patients received BLI801 laxative and 19 received placebo. The sample sizes were selected to allow for qualitative comparisons between groups and with prior data. The study was not powered to detect statistically significant differences between treatment groups.

The primary endpoint of the study was the percentage of patients experiencing a bowel movement within 3 hours of the first study medication dose. The co-primary endpoint was the percentage of subjects with a successful treatment week, defined as not meeting ROME III criteria at the end of the treatment week. Seventy percent of subjects that received BLI801 had a bowel movement within 3 hours versus 53% of placebo subjects. With respect to treatment success, 69.7% of BLI801 subjects did not meet the ROME III definition of constipation by the end of the study versus 26.3% of placebo recipients. There was no evidence of tachyphylaxis.

There were no statistically significant differences between BLI801 Laxative and placebo with respect to treatment emergent adverse events. Also, there were no significant differences seen for the expected patient reported symptoms of gas and cramping (rated using a five point scale ranging from “1=None” to “5=Severely Distressing”). More subjects experienced watery stools in the BLI801 group, which is expected due to its laxative effect. There were no serious adverse events during the study and no on-study deaths. Laboratory results showed no differences between BLI801 Laxative and Placebo for serum chemistry data, including sulfate.

This study supports the conclusion that BLI801 laxative is effective both as a one day “on demand” treatment for constipation as well as a laxative for daily use.

Sulfate Laxative Formulation

Sulfate solutions were evaluated in a study (Example 1) comparing sulfate salt compositions containing 43.8 mmol sulfate salts (4.2 g total sulfate) or 80 mmol sulfate salts (7.7 g total sulfate) to 10 mg bisacodyl (008-211, see, Example 2 above). The compositions were tested in seven normal volunteers for efficacy to induce a rapid, controlled bowel movement without significant gains or losses of electrolytes. Volunteers were given a single dose of laxative in an in-patient setting during which all stool and urine were collected for 24 hours after laxative administration.

A sulfate formula containing 43.8 mmol sulfate salts induced less stool and fewer bowel movements over a 12 hour period than 10 mg bisacodyl. Sulfate formulas containing 80 mmol sulfate salt induced about the same amount of stool and bowel movements as 10 mg bisacodyl, although the stools had a higher water content and tended to be more liquid. The 80 mmol sulfate salt formulation, but not the 43.8 mmol sulfate salt formula, had a much faster time of onset inducing a bowel movement in less than three hours in all subjects. Only one bisacodyl volunteer had a bowel movement in less than three hours. No study volunteer reported incontinence in association with any of the test laxatives. Gastrointestinal symptoms were similar between the 10 mg bisacodyl and the sulfate formulations and no unexpected adverse events were reported. A formulation providing a 3:1 ratio of sodium to potassium minimized sodium and potassium electrolyte absorption or loss from the laxative and was considered appropriate for further study in constipated patients at a dose of about 60 mmol sulfate salt (5.8 gr total sulfate). This formulation was named BLI801 laxative.

### Study Objective

The purpose of this study was to evaluate the efficacy and tolerability of the BLI801 laxative formulation versus placebo in constipated outpatients.

### Study Plan

In this double blind, placebo controlled study, BLI801 Laxative or similarly flavored placebo were provided to subjects meeting a definition of constipation according to ROME III criteria (Longstreth et al., Gastroenterol (2006) 130:1480-1491). In addition, study subjects must have reported fewer than 3 satisfactory BMs during a 7 day Screening Period. Treatment assignments were determined according to a computer generated randomization schedule where subjects were sequentially assigned pre-randomized kits of BLI801 Laxative or placebo in a 2:1 ratio. Subjects self-administered their liquid study medication starting with a dose containing about 6 grams sulfate. If the subject did not experience a BM within 3 hours of taking the first dose, a second dose of study medication (also 6 grams of sulfate) was permitted. Study subjects continued to take a single dose of their study medication daily for 6 additional days. Study subjects rated each BM in a paper diary for satisfaction and completeness. They also rated the urgency associated with their BM, gas, cramping, ROME criteria, and Bristol stool form rating. If study subjects consistently experienced diarrhea and/or loose stools, they were allowed to reduce the daily dose by 25%.

At the end of the study, study subjects were asked to complete a questionnaire indicating their preference for their study medication relative to their previous laxative experience.

Blood samples were collected and a physical examination was performed at the start and end of study.

Study Design: Choice of Control Groups

This study was designed as a double blind, parallel treatment study in adult constipated outpatients to assess the efficacy of BLI801 Laxative as a one day “on demand” therapy. In addition, the study was intended to evaluate BLI801 Laxative effectiveness as a daily laxative. A “completed” subject is defined as one who took the study treatment and completed Visits 2 and 3.

BLI801 Laxative was provided as a liquid preparation. BLI801 Laxative and a similar tasting placebo solution were provided in identically labeled bottles to preserve blinding.

Duration of the Study

The after Visit 1, subjects began a 7 day Screening period to confirm their constipation status. Qualifying subjects were randomized at Visit 2 and began taking their assigned study medication for 7 consecutive days, starting the day after Visit 2. Subjects were instructed to return to the clinic for Visit 3 after Treatment Day 7.

Selection of Study Population

A. Inclusion Criteria:

Male and female outpatients were enrolled according to the criteria listed below.
1. At least 18 years of age.
2. Constipated, defined by the following ROME III definition (Longstreth et al. (2006) Gastroenterology 130(5):1480-1491):
   A. Subject must report 2 or more of the following:
      a. Straining during at least 25% of defecations
      b. Lumpy or hard stools in at least 25% of defecations
      c. Sensation of incomplete evacuation for at least 25% of defecations
      d. Sensation of anorectal obstruction/blockage for at least 25% of defecations
      e. Manual maneuvers to facilitate at least 25% of defecations
         (e.g., digital evacuation, support of the pelvic floor)
      f. Fewer than 3 defecations per week
   B. Loose stools are rarely present without the use of laxatives
   C. There are insufficient criteria for IBS

Criteria A, B and C must be fulfilled for the last 3 months with symptom onset at least 6 months prior to diagnosis.
3. Otherwise in good health, as determined by physical exam and medical history
4. If female, and of child-bearing potential, is using an acceptable form of birth control (hormonal birth control, IUD, double-barrier method, depot contraceptive, sterilized, abstinent, or vasectomized spouse)
5. Negative urine pregnancy test at screening, if applicable
6. In the Investigator's judgment, subject is mentally competent to provide informed consent to participate in the study

B. Exclusion Criteria:

Subjects who met any of the following criteria were excluded from the study:
1. Subjects with known or suspected ileus, gastrointestinal obstruction, gastric retention, bowel perforation, toxic colitis, toxic megacolon.
2. Subjects taking laxatives or prokinetic agents that refuse to discontinue these treatments from Visit 1 until after completion of Visit 3.
3. Subjects who are pregnant or lactating, or intend to become pregnant during the study
4. Subjects of childbearing potential who refuse a pregnancy test.
5. Subjects who are allergic to any BLI801 component (sodium sulfate, potassium sulfate, citric acid, malic acid, sucralose or Dragon Fruit Flavor).
6. Subjects following a physician prescribed sodium restricted diet.
7. Subjects taking narcotic analgesics or other medications known to cause constipation, including, but not limited to, those listed in Appendix 16.1.10.
8. Subjects who, in the opinion of the Investigator, should not be included in the study for any reason, including inability to follow study procedures.
9. Subjects who have participated in an investigational clinical, surgical, drug, or device study within the past 30 days.
10. Subjects with an active history of drug or alcohol abuse.
11. Subjects who withdraw consent at any time prior to completion of Visit 1 procedures

C. Pregnancy:

Subjects that are female and of childbearing potential had a urine pregnancy test done at screening. A positive result ruled out the participation of the patient in the study.

Female study subjects were surgically sterilized or using oral contraceptives, depot contraceptives, double-barrier method, intrauterine device, or testified that she was monogamous with a vasectomized partner, or practiced abstinence and continued to do so during the duration of study. Women with a history of bilateral tubal ligation were not considered of childbearing potential and were not required to have a urine pregnancy test at screening.

Oral contraceptives, hormone implants, and injections were only considered effective if started at least 1 month before the study, and continued until 1 month after the study ended.

Menopausal status was defined when menses have been absent for 12 months in a woman of appropriate age (usually 45 to 55 years) who had no other suspected or identified cause of amenorrhea.

If a subject became pregnant during the study, the subject was removed from the study and followed until one month after the end of the pregnancy.

D. Concomitant Medications:

The use of concomitant medication was recorded from 7 days prior to Visit 1 until the end of the study. Subjects enrolled in this study were not permitted to take any laxatives, whether prescription or over-the-counter, from Visit 1 until after completion of Treatment Day 7. Any restricted laxative use during the study resulted in termination of the subject's participation.

### Study Procedures

Primary Endpoint

The primary endpoint of the study was assessed on the basis of a binary outcome of overall treatment success or failure on the first day of the study where a successful treatment is defined as a treatment that induces one or more bowel movement within 3 hours of the first study medication dose. A failed treatment is defined as a treatment that does not induce at least one bowel movement within 3 hours after drug administration. The co-primary endpoint was the percentage of patients with a successful treatment week, defined as no longer meeting ROME III criteria at the end of the treatment week.

Secondary Endpoint

Secondary endpoints included the following: percentage of subjects that are treatment successes for both Day 1 and treatment week; percentage of patients experiencing a satisfactory and/or complete bowel movement (BM) within 3 hours of the first dose, time of first BM, BM frequency, assessment of common laxative symptoms (nausea, cramping, stomach bloating, urgency), stool amount and form (e.g., Bristol Stool Form score). Safety was assessed using treatment emergent adverse event and laboratory data.

Visit 1

Following the informed consent process, study subjects signed a consent form. Subject's medical history and concomitant medications were reviewed. A baseline physical examination was performed and vital signs taken, including: blood pressure, pulse, temperature, height and weight. A urine pregnancy test will be performed for female patients of childbearing potential. Subjects that meet all inclusion/exclusion criteria entered into the 7-day Screening Period which began the day after Visit 1. Starting on the day of Visit 1, subjects discontinued any laxative use, with the exception of fiber. Subjects taking fiber must have been on a consistent dose for at least 7 days leading up to Visit 1. A Screening Diary was dispensed to subjects to report their BM experiences.

Visit 2

Subjects returned to the study center as soon as possible after the 7 day Screening Period for Visit 2. Screening Diaries were reviewed by site personnel. Subjects who did not return a Screening Diary or who had taken a laxative during the Screening Period were discontinued. During Visit 2, subjects were provided with instructions on how to prepare the study treatment. A total of 8 bottles were dispensed per subject (one for each day of the Treatment period plus a second dose for Treatment Day 1 for patients who did not have their first bowel movement within 3 hours). BM Entry Criterion—to be eligible for randomization, a subject must have reported fewer than 3 satisfactory BMs during the 7 day Screening Period.

Visit 3

Subjects returned to the study center for Visit 3 one day after completing Day 7 of treatment. Study subjects had a physical examination performed, vital signs were obtained and the subject was queried for occurrence of adverse events and changes in concomitant medications. Subjects brought back the Treatment Diary and used study medication. Study personnel reviewed the Treatment Diary for completeness in the presence of the subject so that any missed responses were captured.

A blood sample was collected for analysis of serum chemistry. A second serum sample was collected for analysis of serum sulfate at BioAssay Systems (Hayward, Calif.).

Method of Assigning Subjects to Treatment Groups

Subjects were randomly assigned in a 2:1 ratio (BLI801:Placebo) within each participating site. The randomization schedule for this study was created by StatNet Statistical Services Network and was constructed using random blocks of 3 balanced treatment assignments. The randomization schedule was implemented prior to kit distribution to the site. Following receipt of a sequential series of drug kits, site personnel dispensed the lowest numbered kit available to subjects that met eligibility criteria in order to maintain the randomization schedule.

Disposition of Subjects

This study was conducted at 4 study centers. 64 subjects were screened and 52 were randomized and dispensed study medication. One female subject receiving placebo discontinued from the study due to an adverse event (cramping and flatulence).

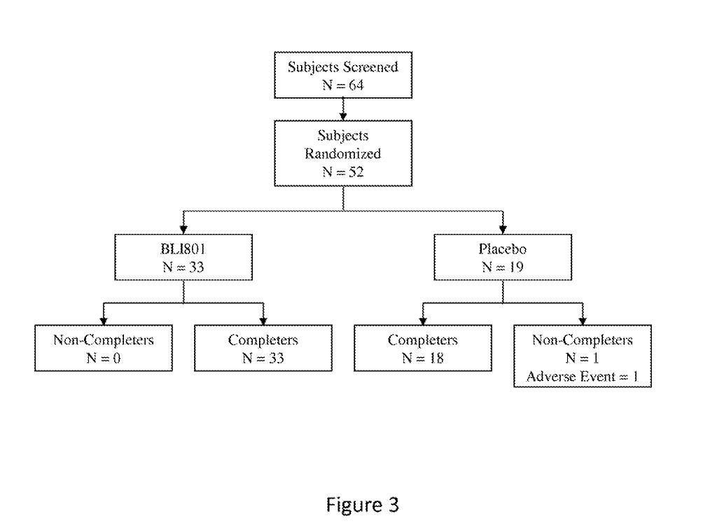

The disposition of all study subjects is shown in FIG. 3.

Treatments

A. Treatments Administered:

BLI801 Laxative was provided in bottles containing sulfate salts (as shown below) with flavoring ingredients in approximately 2.5 ounces (about 75 ml) of solution:

TABLE 8
BLI 801 Laxative

| Component | Amount |
|---|---|
| (anhydrous equivalents) | (g) |
| Na₂SO₄ | 6.65 g |
| K₂SO₄ | 2.71 g |

Each bottle contained a single day's dose of 62 mmol sulfate salts or 6.0 grams total sulfate or placebo (a flavored salt blend with no active ingredients) with a clinical label containing a caution statement, study code, study sponsor and kit number. The study medications were provided in identical packaging.

Placebo solution was provided in identically label bottles containing 3.8 g sodium chloride with flavoring ingredients in approximately 2.5 ounces (about 75 ml) of solution. Subjects were instructed to mix each bottle with water to a total volume of 8 oz. Each study subject received a total of 8 bottles. One bottle was to be taken on each day of the treatment period. A second dose, to be taken on Treatment Day 1, was permitted if the subject did not have a bowel movement within 3 hours following their initial dose. Subjects were instructed to bring he used drug components when they returned for Visit 3.

B. Treatment Day 1:

Subjects completed the Treatment Diary starting on the morning of Treatment Day 1. All food and beverages ingested on Treatment Day 1 were documented on the Treatment Diary. All bowel movements, including those occurring prior to the first dose were documented on the Treatment Diary.

Subjects mixed one bottle of the assigned medication with water to 8 total ounces using the graduated measuring cup provided by the site. Between 6:00 A.M.-8:00 P.M. subjects ingested the first dose of the solution. Doses taken outside the window were considered protocol violations.

If the subject did not have a BM within 3 hours, a second bottle was taken (3-4 hours from the prior dose). A second daily dose was allowed only on Treatment Day 1.

C. Treatment Days 2-7:

Subjects consumed 1 dose per day (one bottle each day mixed with water to 8 ounces) on Treatment Days 2 through 7. These doses were taken at any time between the hours of 6:00 A.M.-8:00 P.M. Subjects were encouraged to consume each dose at approximately the same time of day. Completion of the Treatment Diary continued until the end of Treatment Day 7.

D. Dose Reduction:

Subjects who consistently experienced diarrhea and/or loose stools, were be allowed to reduce the daily dose by 25%. The entire bottle of solution was mixed with water to 8 ounces. Subjects then consumed only 6 ounces of the mixed solution. Subjects documented their dose reduction in the Treatment Diary.

E. Day 8-Visit 3:

Subjects will returned to the study center for Visit 3 one day after completing Day 7 of treatment. Subjects scheduled their final visit 8 days after the date of Visit 2. Visits conducted outside this window were considered protocol violations.

Study subjects had a physical examination performed, vital signs were obtained and the subject were queried for occurrence of adverse events and changes in concomitant medications. Subjects brought back the Treatment Diary and used study medication. Study personnel reviewed the Treatment Diary for completeness in the presence of the subject so that any missed responses could be captured.

A blood sample was collected for analysis of serum chemistry. A second serum sample was collected for analysis of serum sulfate.

F. Selection of Dose in the Study:

The 6 g sulfate dose of BLI801 Laxative was based on previous studies as discussed above.

G. Selection and Timing of Dose for Each Subject:

Selection and timing of dose for BLI801 was based upon results of previous studies as discussed above. These previous studies had explored the efficacy of a single dose of BLI to induce a rapid (within 3 hours) bowel movement. This dose was utilized in the current study. The current study was also intended to assess the efficacy of the 6 g sulfate dose for repeat dosing over a seven day treatment period. The timing of the daily dose for the repeat dosing was not expected to significantly affect study results

H. Blinding:

Study medications were provided in identically labeled bottles to preserve blinding. Placebo was formulated to have a similar taste to BLI801 Laxative.

I. Prior and Concomitant Therapy:

Subjects were not permitted to take laxatives or prokinetic agents during the study. Narcotic analgesics or other medications known to cause constipation were also prohibited.

J. Treatment Compliance:

On their final study visit (Visit 3), subjects were instructed to return all used and unused drug supplies to the clinic. Staff members performed drug accountability by measuring and any remaining amounts of unused study medication returned, and by querying study subjects for compliance.

Table 9 depicts the flow of study procedures at each visit.

TABLE 9

| | | Screening | | Treatment | |
|---|---|---|---|---|---|
| Procedures | Visit 1 | Days 1-7 | Visit 2 | Days 1-7 | Visit 3 |
| Informed Consent | X | | | | |
| Inclusion/Exclusion Criteria Review | X | | | | |
| Medical History | X | | | | |
| Physical Examination | X | | | | X |
| Vital Signs¹ | X | | | | X |
| Review of Concomitant Medication | X | | | | X |
| Urine Pregnancy Test (if applicable) | X | | | | |
| Dispense Subject Diary | X | | X | | |
| Instruct Subject | X | | X | | |
| Subject Completes Diary | | X | | X | |
| CRC Reviews Diary | | | X | X | |
| Dispense Drug (if subject is eligible) | | | X | | |
| Blood Sample for Chemistry Testing | | | X | X | |
| Blood Sample for Serum Sulfate | | | | | X |
| Testing | | | | | |
| Subject Completes Preference | | | | | X |
| Questionnaire | | | | | |
| Subject Takes Study Drug | | | | X | |
| Drug Accountability | | | | | X |
| Assess Safety | | | | | X |

¹blood pressure, pulse, temperature, height and weight.

### Assessments

Efficacy Measurements

Study subjects were instructed to report their BM experiences on paper diaries, which were completed during both the Screening and Treatment Periods. For each BM subjects were asked to record the date and time; to rate any gas or cramping (using a 5 point scale where 0=none and 4=extreme); stool amount or urgency (using a 3 point scale where 1=a little and 3=a lot); if the BM was satisfactory, complete, straining, lumpy or hard, anorectal obstruction, or if they required manual maneuvers to promote defecation (all yes/no answers). In addition, subjects were required to rate each BM according to the Bristol Stool Scale as follows:
1=Separate hard lumps like nuts (difficult to pass)
2=Sausage shaped but lumpy
3=Like a sausage but with cracks on its surface
4=Like a sausage or snake, smooth and soft
5=Soft blobs with clear-cut edges (passed easily)
6=Fluffy pieces with ragged edges, a mushy stool
7=Watery, no solid pieces, entirely liquid

The Treatment Diary also prompted study subjects to record the date, time and amount of each dose taken as well as food and fluid intake on Treatment Day 1. Subjects were also prompted to record any non-study laxative use.

At Visit 3, study subjects completed a preference questionnaire asking them to compare their experience with the study medication to their past laxative experience. Subjects answered the following questions:

| 1. Please list any OTC or prescription laxatives that you | | |
|---|---|---|
| have taken over the past year. | | |
| 2. Please answer the following questions comparing your | | |
| study medication to your past laxative: | | |
| If Prior Laxative, please identify brand. | | |
| Which was easier to take? | | |
| Study Laxative | Prior Laxative | No Difference |
| Which made you have a bowel movement faster? | | |
| Study Laxative | Prior Laxative | No Difference |
| Which allowed you the most control of bowel movements? | | |
| Study Laxative | Prior Laxative | No Difference |

Safety Measurements

Safety assessments included adverse event monitoring as well as pre and post treatment period physical examination (Visits 2 and 3). Unsolicited verbal expressions of severe symptoms by the subject were recorded as adverse events.

An adverse event for “Cramping” or “Gas”, was completed if the event was rated by the subject as “extreme” (score=4) on their Treatment Diary. If multiple episodes of cramping and/or gas are reported on the Treatment Diary in a given day, these were represented as one adverse event on the case report form with severity reflecting the most severe episode for that day.

Blood samples were collected at Visits 2 and 3 for chemistry analysis. Serum samples were collected at Visit 3 for testing of sulfate. Serum sulfate testing was performed by BioAssay Systems (Hayward, Calif.).

Data Quality Assurance

One hundred percent source document verification was performed on all study data points. Case Report Forms were processed and any discrepancies identified during the data management procedures were resolved via Data Clarification Forms which were completed and signed by study investigators.

Following database closure, a quality control audit was performed comparing database output to case report forms for 100% of study data points for all subjects. All discrepancies identified during this audit were resolved with investigators prior to locking the database.

Adverse Event Definition and Reporting

An Adverse Event (AE) was any untoward medical occurrence in a patient or clinical investigation subject administered a pharmaceutical product and that did not necessarily have a causal relationship with this treatment. An AE can therefore be any unfavorable and unintended sign (including an abnormal laboratory finding), symptom, or disease temporally associated with the use of a medicinal (investigational) product.

An adverse event for “Cramping” and “Gas”, was completed when rated by the subject as “extreme” (score=4) on their Treatment Diary. If multiple episodes of cramping and/or gas were reported on the Treatment Diary in a given day, these were represented as one adverse event on the case report form with severity reflecting the most severe episode for that day. In addition, unsolicited verbal expression of severe symptoms by the subject was recorded as adverse events regardless of the score on the Treatment Diary.

Adverse event collection was based on treatment emergence. Investigators were required to report all adverse events beginning on Treatment Day 1 and concluding with the end of study participation. Patients were instructed to report promptly adverse events to the Investigator. The Investigator recorded date/time of report, date/time of onset, description of the adverse event, severity of adverse event, action(s) taken regarding treatment of the event, action(s) taken regarding study participation, duration of adverse event, and the Investigator's assessment of relationship of adverse event to study treatment.

The Investigator assessed the severity of each adverse event using the categories as provided in Table 10:

TABLE 10

| Grade | Severity | Description |
|---|---|---|
| 1 | Mild | Barely noticeable, does not influence |
| | | functioning |
| | | Causing no limitations of usual activities |
| 2 | Moderate | Makes participant uncomfortable, |
| | | influences functioning |
| | | Causing some limitations of usual activities |
| 3 | Severe | Severe discomfort, treatment needed |
| | | Severe and undesirable, causing inability |
| | | to carry out usual activities |
| 4 | Life | Immediate risk of death |
| | threatening | Life threatening or disabling |
| 5 | Fatal | Causes death of the participant |

The Investigator assessed the relationship to study drug for each adverse event using the categories as provided in Table 11:

TABLE 11

| Categories of | |
|---|---|
| Attribution: | Description |
| UNRELATED | There is no evidence of any causal relationship. |
| POSSIBLE | There is some evidence to suggest a causal |
| | relationship (e.g., the event occurred within a |
| | reasonable time after administration of the |
| | trial medication). However, the influence of |
| | other factors may have contributed to the event |
| | (e.g., the patient's clinical condition, other |
| | concomitant events). |
| PROBABLE | There is evidence to suggest a causal relationship, |
| | and the influence of other factors is unlikely. |
| DEFINITE | There is clear evidence to suggest a causal relationship, |
| | and other possible contributing factors can be ruled out. |

In Phase III studies, adverse events associated with Oral Sodium Sulfate administered at doses consistent with colonoscopy preparation have included nausea, vomiting, abdominal cramping and bloating. These adverse reactions were transient and subsided rapidly.

Serious Adverse Reactions and Discontinuation of Study

A Serious Adverse Event (SAE) was any untoward medical occurrence that results in at least one of the following outcomes:
Results in death
Is life-threatening
Requires inpatient hospitalization or prolongation of existing hospitalization
Results in persistent or significant disability/incapacity
Is a congenital anomaly/birth defect
Requires medical or surgical intervention to prevent permanent impairment or damage

SAE collection coincided with the patient providing informed consent to participate in the study and concluded 30 days after the end of study participation. Should a serious and/or unexpected adverse event occur, the Investigator must notify Braintree Laboratories immediately. The Investigator made a decision regarding continuing study participation, and requested input from the sponsor. The Investigator was responsible for recommending or providing the subject with appropriate medical therapy. All subjects experiencing serious adverse events were followed until satisfactory resolution occurs.

### Analysis Methods

Study Endpoints

There were two primary efficacy endpoints. The first efficacy endpoint was assessed on the basis of a binary outcome of overall treatment success or failure on the first day of the study where a successful treatment was defined as a treatment that induced one or more bowel movements within 3 hours after drug administration. A failed treatment was defined as a treatment that does not induce at least one bowel movement within 3 hours after drug administration. The second primary efficacy endpoint was the percent of subjects with a successful treatment week, defined as not meeting ROME III criteria at the end of the treatment week.

Secondary endpoints included the following: the proportion of study subjects that subjects that were treatment successes for both Day 1 and the treatment week; the proportion of subjects experiencing a satisfactory BM within 3 hours; the proportion of subjects experiencing a complete BM within 3 hours; the proportion of subjects experiencing a complete/satisfactory BM within 3 hours; the time of first BM following Dose 1; mean BM symptom scores (e.g., cramping, gas, urgency); the mean BM stool amount; the mean Bristol Stool Form score; the number of BMs per day; the number of complete/satisfactory BMs per day; the number of BMs per week; and the number of complete/satisfactory BMs per week. Serum sulfate concentrations and pharmacokinetic parameters were tabulated and summarized with descriptive statistics (N, mean, SD, SEM, minimum, and maximum).

Determination of Sample Size

The planned enrollment for this pilot study was forty-five subjects, randomly assigned to either BLI801 or placebo in a ratio of 2:1 (30 BLI801 subjects: 15 placebo subjects). The sample size will allow for qualitative comparisons of efficacy and safety data between groups and with previous studies using prior sulfate formulations as well as other approved laxatives.

Changes in the Conduct of the Study or Planned Analyses

One protocol amendment was implemented after initiation of the study. The protocol was amended to change the serum sulfate testing laboratory from WIL Research (Ashland, Ohio) to Bioassay Systems (Hayward, Calif.). No other changes to the study design or statistical analysis plan were implemented during or after this study.

Protocol Deviations

No significant protocol deviations occurred during this study. All subjects were considered evaluable for efficacy and safety analyses.

### Efficacy Results

Data Sets Analyzed

All enrolled subjects that took study medication (n=52) were included in the safety and efficacy analysis.

Demographic and Other Baseline Characteristics

No statically significant demographically related differences were noted between the treatment groups. The treatment groups were similar with respect to age, gender, racial distribution and baseline weight. The average age of study subjects was about 42 years, ranging from 19 to 82 years of age. About 87% of subjects were Caucasian and 8% were African American. Study subjects weighed an average of about 169 lbs.

The study population demographics are summarized in Table 12, below.

TABLE 12
Study Demographics

| | BLI801 | | Placebo | | p¹ |
|---|---|---|---|---|---|
| Age (years)² | | | | | |
| n | 33 | | 19 | | 0.940 |
| Mean (SD) | 42.1 | (17.9) | 42.5 | (14.3) | |
| Gender | | | | | |
| Male | 7 | (21%) | 2 | (10%) | 0.458 |
| Female | 26 | (79%) | 17 | (90%) | |
| Race³ | | | | | |
| Caucasian | 30 | (91%) | 15 | (79%) | 0.308 |
| A. Am. | 3 | (9%) | 1 | (5%) | |
| Other | 0 | | 2 | (11%) | |
| Weight (lbs) | | | | | |
| Mean (SD) | 171.9 | (36.1) | 164.3 | (39.3) | 0.486 |

¹P-value from exact Chi-Square test for the categorical variables and from an ANOVA with term for treatment for the continuous variables
²Age is calculated using of date of birth and screening visit (Visit 1) date.
³Percentage for race does not equal 100% since Hispanic or Latino subjects may not have reported a race.
SD = standard deviation;
A. Am. = African American

Measurements of Treatment Compliance

Following completion of their randomized treatment, subjects reported to their study center (Visit 3) where they returned study drug materials which were reviewed for treatment compliance.

Compliance was based on review of used drug materials and subject interview. Compliance with dosing during the 7 day treatment period was excellent. One hundred percent of BLI801 subjects completed all study doses compared to 95% of placebo subjects (1 placebo subject discontinued early due to adverse events).

Efficacy Results and Tabulations

A. Analysis of Efficacy—Primary Endpoints:

A higher percentage of subjects that received BLI801 Laxative, 69.7% (23/33), had their first BM within 3 hours of their first dose ingestion versus 52.6% (10/19) of placebo subjects. This result appears to be reflected in the average time to bowel movement on the first day of treatment where the BLI801 Laxative group mean time was 2.7 hours (SD=2.8 hrs) versus 11.0 hours (SD=20.9 hrs) for placebo. Twenty one percent (7/33) of BLI801 Laxative subjects required a second dose of study medication versus 53% (10/19) of placebo recipients.

A larger proportion of BLI801 Laxative subjects (69.7%, 23/33) did not meet ROME III criteria by the end of treatment compared to placebo subjects (26.3%, 5/19). This difference was statistically significant (p<0.01).

B. Analysis of Efficacy—Secondary Endpoints:

Summary data for the first day of treatment is shown below in Table 13:

TABLE 13
First Day Measures

| Measure (SD) | BLI801 | Placebo | P |
|---|---|---|---|
| Number of BM | 2.8 (1.8) | 1.2 (0.8) | 0.004 |
| Number of Complete & | 1.8 (1.9) | 0.6 (0.7) | 0.058 |
| Satisfactory BM | | | |
| Mean Bristol Score¹ | 5.0 (1.9) | 3.6 (1.5) | 0.021 |

¹Bristol Stool Score: 7 point scale where 1 = hard lumps, to 7 = watery

As shown in the table, on their first day of treatment, subjects that received BLI 801 Laxative had about 3 bowel movements of which approximately two were considered to be complete and satisfactory.

Over the 7 day treatment period, the number of subjects that had a BM within 3 hours following dose ingestion remained relatively constant as shown in Table 14. The predictable performance of BLI801 Laxative is further demonstrated by its reproducible result within the same individual patient over the course of the treatment week. Sixty one percent of BLI801 Laxative subjects had a BM within 3 hours of dosing on 4 or more treatment days (>50%) compared to 21% of placebo subjects.

TABLE 14
Percent (N) of Subjects BM less than 3 hrs

| | Day 1 | Day 2 | Day 3 | Day 4 | Day 5 | Day 6 | Day 7 |
|---|---|---|---|---|---|---|---|
| BLI801 | 70% (23) | 55% (18) | 58% (19) | 58% (19) | 67% (22) | 55% (18) | 70% (23) |
| n = 33 | | | | | | | |
| Placebo¹ | 53% (10) | 32% (6) | 44% (8) | 17% (3) | 39% (7) | 33% (6) | 33% (6) |
| n = 19 | | | | | | | |

¹One placebo subject withdrew after Day 2. Percents for Days 3-7 are based on an n = 18.

Relevant secondary efficacy measures for the 7 day treatment period are summarized in Table 15, below:

TABLE 15
Mean Ratings and Scores for Treatment Week

| Measure (mean) | BLI801 | Placebo | p |
|---|---|---|---|
| Stool Amount¹ per BM | 2.1 (0.4) | 1.8 (0.3) | 0.019 |
| Bristol Rating² per BM | 5.4 (1.4) | 3.3 (1.4) | <0.001 |
| BM/day | 2.2 (1.2) | 1.0 (0.6) | <0.001 |
| Number of Complete | 1.7 (1.3) | 0.5 (0.4) | 0.001 |
| BM/day | | | |
| Number of Satisfactory | 1.6 (1.3) | 0.4 (0.5) | 0.001 |
| BM/day | | | |
| Number of Satisfactory | 1.5 (1.3) | 0.4 (0.5) | 0.004 |
| & Complete BM/day | | | |

¹Stool Amount: 3 point scale 1 = a little, 2 = to 3 = a lot.
²Bristol Stool Score: 7 point scale where 1 = hard lumps, to 7 = watery.

All of the secondary measures were highly statistically significant favoring BLI801 Laxative over placebo for the treatment week.

C. Efficacy Conclusions:

BLI801 treatment resulted in rapid stool elimination on the first day of treatment in most subjects. Relative to placebo, a majority of these initial stools were characterized as softer, complete and satisfactory. Secondary measures for the 7 day treatment period were equally successful where stools continued to be softer, more frequent and usually “complete and satisfactory”. BLI801 Laxative has been shown to be effective with respect to rapid onset of action and reliable results over a 7 day treatment period.

### Safety Evaluation

Thirty three subjects consumed a daily dose of BLI801 Laxative containing about 6 grams of sulfate for 7 days.

Adverse Events

It is useful to consider the number of subjects each day that reported Bristol Stool Scale scores of 6 or 7 each day of treatment. These scores (where 6=Fluffy pieces with ragged edges, a mushy stool; and 7=Watery, no solid pieces, entirely liquid) may be indicative of overly effective treatment. This data is shown below in Table 16.

TABLE 16
Percent (N) of Subjects with Bristol Scores of 6 and 7

| | Day 1 | Day 2 | Day 3 | Day 4 | Day 5 | Day 6 | Day 7 |
|---|---|---|---|---|---|---|---|
| BLI801 | 64% (21) | 64% (21) | 61% (20) | 61% (20) | 64% (21) | 61% (20) | 58% (19) |
| n = 33 | | | | | | | |
| Placebo | 26% (5) | 11% (2) | 11% (2) | 6% (1) | 6% (1) | 6% (1) | 11% (2) |
| n = 19 | | | | | | | |

As shown in the table, subjects taking BLI801 Laxative were significantly more likely to have reported Bristol Stool Scale scores of 6 or 7 (p<0.05 for all days). This effect was consistent throughout the 7 day treatment period, demonstrating that repeated exposure to BLI801 Laxative does not result in tachyphylaxis.

Seven subjects reported frequent watery stools (>3 stools per day with Bristol score=7) in the BLI801 Laxative group. Three of these subjects (1015, 1022, 1031) chose not to reduce their dose as allowed per protocol and continued to report frequent watery stools to Day 7. Three (1014, 2008, 2014) reduced their doses within the first two days of treatment and no longer reported frequent watery stools by the end of study. Only one patient (2002) reduced their dose and still experienced frequent watery stools at Day 7.

Because of the high percentage of subjects experiencing mushy and watery stools in the BLI801 Laxative group, further investigation of lower sulfate doses is warranted if the laxative is developed as a one-week therapy. Furthermore, this effect was maintained after 48% of BLI801 subjects reduced their dose from 6 g to 4 g during the Treatment Week (compared to 5% of placebo subjects).

Safety Conclusions

Thirty-three constipated subjects were treated with BLI801 Laxative for 7 days. The laxative was well tolerated with few side effects other than loose stools. Treatment emergent adverse events were infrequent, with no significant differences detected between the two treatments. There were no untoward effects observed for physical examination or blood analyte measures, including serum sulfate.

Although loose stools are to be expected from laxative use, the frequency of such reports (in the form of Bristol Stool Scale scores of 6 and 7) suggests that a reduced dose of BLI801 would be more appropriate, particularly for repetitive use.

### Conclusion

This double blind, multicenter study compared BLI801 Laxative to a similarly flavored placebo solution in constipated outpatients. BLI801 Laxative was highly successful in inducing bowel movements within 3 hours of ingestion. In addition, more subjects taking BLI801 Laxative did not meet ROME III constipation criteria than subjects receiving placebo over the 7 day treatment period. There was no evidence of tachypylaxis.

BLI801 Laxative was well tolerated. There were no differences in treatment emergent adverse effects, although subjects taking BLI801 Laxative tended to have more gastrointestinal symptoms such as flatulence. As might be expected for a laxative, patient scoring of stool consistency (using the Bristol Stool Scale) showed numerous instances of loose stools, even on the first day of use. This suggests that the dose could be reduced without adversely affecting efficacy.

There were no on study deaths or serious, unexpected adverse reactions.

### Example 3: A Safety and Efficacy Evaluation of BLI801 Laxative in Constipated Adults

### Introduction

Example 1 describes formulation studies in normal volunteers demonstrated that a formulation containing 3:1 sodium sulfate to potassium sulfate minimized sodium and potassium gains or losses from stool relative to 10 mg bisacodyl. This study also showed that a dose containing about 80 mM (7.7 g) sulfate reliably yielded a bowel movement within about 3 hours following ingestion of the laxative. A lower dose of 43.8 mM (4.2 g) was less effective. This formulation was named BLI801.

A preliminary clinical study in 52 patients meeting ROME III constipation criteria (Example 2) showed that about 70% of patients receiving a daily BLI801 dose containing 62 mM (about 6 g) sulfate salts experienced a bowel movement within 3 hours of laxative ingestion versus 53% of patients that received placebo. In addition, 70% of study subjects no longer met ROME III constipation criteria after 7 days of treatment versus only 26% of patients receiving placebo. There were no differences between BLI801 laxative and placebo in terms of treatment emergent adverse events and assessment of serum chemistry similarly showed no statistically significant differences between BLI801 laxative and placebo with no clinically significant changes in serum sodium and potassium.

A second Phase II study to determine if constipated patients would prefer “on-demand” use of BLI801 laxative rather than daily use was therefore desired.

### Methods

This was a randomized, double blind study in 60 adult outpatients who met ROME III criteria for constipation. The protocol was not designed to determine statistically significant differences but rather for qualitative differences to help inform subsequent studies.

At their first visit, study subjects completed quality of life questionnaires (PAC-QOL and PAC-SYM) and provided a blood sample for chemistry analysis. The validated questionnaires may be found in Marquis, P. et al. (2005) Scand J Gastroenterol. 40(5):540-51 and Frank, L. et al. (1999) Scand J Gastroenterol. 34(9):870-7. To be eligible for treatment study subjects had to have fewer than 3 bowle movements per week during the 14 day baseline period where they received no laxatives. Qualifying study subjects were then treated with BLI801 laxative at either 36.5 mM (3.5 g) sulfate or 52 mM (5.0 g) sulfate for 28 days. For the first 14 days of the study, patients were instructed to take their assigned laxative on an as needed or “on demand” basis. They returned to the clinic after the first 14 day period, where they again completed the quality of life questionnaires and provided a sample of blood for analysis. Study patients then continued to take their assigned BLI801 laxative on an on demand basis for the remaining 14 days of the study whereupon they returned to the clinic, completed the quality of life questionnaires, and provided a sample of blood for analysis

### Results

Sixty constipated patients meeting ROME III criteria completed the protocol. Their average age was 43 years and study participants were predominantly female (88%).

There was no difference between the two dose groups with respect to overall treatment success which was defined as >3 complete spontaneous bowel movements (CSBMs) during a given treatment week with an accompanying increase of at least 1 CSBM from baseline for that week for 3 out of 4 weeks of treatment. Overall, the low dose group had about 35.5% of study subjects with a successful outcome according to this definition versus 38.7% for the higher dose. The weekly results for the two doses are shown below in Table 17.

TABLE 17
Treatment Success by Week

| Week | Low Dose | High Dose |
|---|---|---|
| Week 1 (n %) | | |
| Success | 11 (35.5) | 17 (54.8) |
| Fail | 20 (64.5) | 14 (45.2) |
| Week 2 (n %) | | |
| Success | 12 (38.7) | 12 (38.7) |
| Fail | 19 (61.3) | 19 (61.3) |
| Week 3 (n %) | | |
| Success | 12 (41.4) | 15 (48.4) |
| Fail | 17 (58.6) | 16 (51.6) |
| Week 4 (n %) | | |
| Success | 14 (48.3) | 16 (51.6) |
| Fail | 15 (51.7 | 15 (48.4) |

The higher dose was associated with many more patients having a bowel movement within three hours of dose ingestion (54.8%) than the lower dose (29%) and more reports of treatment emergent diarrhea (19% vs 0%). However, average Bristol stool ratings were similar between the dose groups (average scores about 4). No effect of “on demand” dosing versus historical daily treatment protocols was apparent.

As shown in Tables 18 and 19, patients in both the high and low sulfate dose groups reported improvement in constipation symptoms and quality of life after 28 days of “on demand” treatment.

TABLE 18
Patient Assessment of Constipation
Symptom (PAC-SYM) Summary

| Change in PAC-SYM Score | SULAX | SULAX |
|---|---|---|
| by Domain | (low dose) | (high dose) |
| mean (SD) | n = 31 | n = 31 |
| Abdominal Symptoms | −0.96 (1.2) | −1.03 (1.3) |
| Rectal Symptoms | −0.87 (0.9) | −1.21 (1.1) |
| Stool Symptoms | −1.29 (1.2) | −1.52 (1.3) |
| Overall | −1.07 (1.0) | −1.28 (1.1) |

*Negative values indicate improvement

TABLE 19
Patient Assessment of Quality of Life (PAQ-QOL) Summary

| Change in PAC-QOL Score | SULAX | SULAX |
|---|---|---|
| by Domain | (low dose) | (high dose) |
| mean (SD) | n = 31 | n = 31 |
| Extent/Intensity of Constipation | −1.53 (1.4) | −1.50 (1.5) |
| Effect of Constipation on Daily Life | −0.88 (0.9) | −1.20 (1.1) |
| Feelings Related to Constipation | −0.94 (1.1) | −1.26 (1.0) |
| Life with Constipation | −1.32 (1.4) | −1.30 (1.3) |
| Degree of Satisfaction | 1.54 (1.5) | 1.53 (1.4) |
| Overall | −30.5 (28) | −36.4 (27) |

*Negative values indicate improvement, except for Degree of Satisfaction (+ values = improvement)

No clinically significant changes in laboratory analytes were detected between the low dose and high dose treatment groups. The results for sodium and potassium are shown in Table 20.

TABLE 20
Serum Sodium and Potassium

| Analyte (Mean, SD) | Low Dose | High Dose | p |
|---|---|---|---|
| Sodium (mmol/L) | | | |
| Screening | 140.4 (2.2) | 139.8 (1.8) | |
| Week 4 | 139.9 (2.5) | 139.6 (2.0) | |
| Change | −0.3 (2.7) | −0.2 (2.3) | 0.856 |
| Potassium (mmol/L) | | | |
| Screening | 4.13 (0.4) | 4.04 (0.3) | |
| Week 4 | 4.14 (0.5) | 3.94 (0.4) | |
| Change | 0.03 (0.4) | −0.10 (0.3) | 0.188 |

### Conclusion

This qualitative study showed that both the high and low doses of BLI801 laxative were effective in treating constipation and improving patient quality of life and symptom indicators when used in an “on demand” regimen, although no advantage for “on demand” treatment versus historical results from daily dosing was observed. Although not designed for quantitative differences, the study again showed a dose response relative to patients having a bowel movement within 3 hours of laxative ingestion.

There were no differences in treatment emergent adverse events except for diarrhea which was associated with the higher laxative dose. There were no clinically significant changes in blood analytes detected, particularly for sodium and potassium. Given the efficacy of BLI801 to induce a rapid bowel movement soon after ingestion, it is expected that this composition may be used in difficult to treat groups such as patients with opioid and other drug-induced constipation.

### Example 4: Cross-Study Quality of Life Comparison of BLI801 to MiraLax

A comparison was made to assess patient perceptions of the effect of BLI801 to a popular over-the-counter laxative, MIRALAX. Table 21 provides a summary of the doses and dosing schedules of the constipated subjects enrolled in Study BLI801-202 (described in Example 3) and constipated subjects enrolled in the MIRALAX study (see, DiPalma, et al. (2007) Am. J Gastroenterol 102:1-8) who completed the same Patient Assessment of Constipation (PAC) Symptom and Quality of Life Questionnaire Summary as used in Example 3 (Marquis, P. et al. (2005) Scand J Gastroenterol. 40(5):540-51 and Frank, L. et al. (1999) Scand J Gastroenterol. 34(9):870-7). The results of this comparison are presented in Table 22.

As shown in Table 22, the comparison confirms that, from the study subjects' perspective, BLI801 is equally effective to MIRALAX and by several measures the high dose appears to give better responses for indicators such as “effect of constipation on daily life” and “feelings related to constipation”.

TABLE 21
Cross-study Quality of Life Comparison of BLI801 to MiraLax

| | BLI801 | MIRALAX |
|---|---|---|
| Study Identifier | BLI801-202 | 851-ZCC |
| | (NCT 01687985) | (NCT 00153140) |
| Patient Population | Chronic Constipation | ROME Criteria for |
| | | Constipation at Baseline |
| N | 62 patients | 150 patients |
| Dose | Oral solution | 17 gm powder |
| | 5 g sulfate (high dose) | |
| | 3.5 g sulfate (low dose) | |
| Regimen | On Demand Dosing | Daily Dosing |
| | 28 days | 28 days |

### Example 5: Cross-Study Comparison of BLI801 to Relistor

RELISTOR is a prescription drug (which is intra-abdominally administered) indicated for treatment of constipation associated with opiate use. Such patients tend to be severely constipated. Table 23 provides a comparison of severely constipated BLI801 treated patients from Study BLI801-202 (described in Example 3—defined as patients with 5 or fewer bowel movements in the two week baseline period) to published data for RELISTOR in patients with opiate constipation (ClinicalTrials.gov Identifier: NCT00529087 and Michna, E., et al. (2011) Pain Med. 12(8):1223-30). Table 23 contains comparisons of BLI801 and RELISTOR for key efficacy measures, including onset of action (time to first bowel movement), stool frequency and consistency. The results of these analyses indicate that BLI801 appears to be more efficacious than RELISTOR for all of these key efficacy measures.

### Example 6. Liquid Formulation

A randomized, open-label pilot study was conducted in 60 constipated patients (Braintree Study BLI801-101) using SUPREP. SUPREP was administered at four different dose levels (1.2 g, 3.7 g, 6.2 g and 8.7 grams of sulfate salts) as a single dose of solution to evaluate product efficacy to induce bowel movements within 3 hours of ingestion while minimizing patient discomfort and diarrhea. Since it had been reported that about 4 grams of sulfate are required to have a laxative effect (7), the 1.2 g dose was chosen as a placebo dose. Patient tolerance and safety were expected to be superior to over-the-counter (OTC) stimulant laxatives (e.g. bisacodyl).

As shown in Table 24 below, most patients in the 6.2 g and 8.7 g groups had at least one bowel movement within 3 hours after ingesting their dose of sulfate salts. As expected, the number having a bowel movement within three hours increased with increasing dose.

TABLE 24
Patients with BM < 3 hrs

| Dose | n | # with BM |
|---|---|---|
| 10 ml (1.2 g) | 15 | 6 (40%) |
| 30 ml (3.7 g) | 15 | 8 (53%) |
| 50 ml (6.2 g) | 14 | 12 (86%) |
| 70 ml (8.7 g) | 14 | 10 (71%) |

Table 25 shows a direct relationship between stool amount and consistency, with the 6.2 g and 8.7 g groups experiencing larger bowel movements of softer consistency. As expected, these groups also reported higher Bristol Stool Scores, indicating softer, looser stools.

TABLE 25
Mean BM Amount and Consistency

| | | Stool | Stool | Mean |
|---|---|---|---|---|
| | | Amount¹ | Consistency² | Bristol Stool |
| Dose | n | Score | Score | Scale³ |
| 10 ml (1.2 g) | 15 | 1.5 | 1.5 | 3.0 |
| 30 ml (3.7 g) | 14 | 1.8 | 1.8 | 3.8 |
| 50 ml (6.2 g) | 14 | 2.2 | 2.1 | 4.5 |
| 70 ml (8.7 g) | 15 | 2.1 | 2.2 | 5.0 |

¹Amount: 1 = a little; 2 = some; 3 = a lot
²Consistency: 1 = hard; 2 = soft; 3 = liquid
³Bristol: 1 = separate hard lumps, like nuts - 7 = watery, no solid pieces

No significant adverse events were reported. Based on efficacy and stool form results, a dose of approximately 6.2 grams appears adequate to achieve laxation without eliciting excessive loose stools.

### Example 7. Liquid Sulfate Reformulation

The sulfate solution was reformulated to exclude magnesium sulfate. This formulation was evaluated in a single center study comparing sulfate salt compositions containing 43.8 mM (4.2 g) or 80 mM (7.7 g) sulfate to 10 mg bisacodyl (Baylor Study 008-211). The compositions were tested in seven normal volunteers for efficacy to induce a rapid, controlled bowel movement without significant gains or losses of electrolytes. Volunteers were given a single dose in an in-patient setting during which all stool and urine were collected for 24 hours after laxative administration.

A sulfate formula containing 43.8 mM sulfate induced less stool and fewer bowel movements over a 12 hour period than 10 mg bisacodyl. Sulfate formulas containing 80 mM sulfate induced about the same amount of stool and bowel movements as 10 mg bisacodyl, although the stools had a higher water content and tended to be more liquid. The 80 mM sulfate formulation, but not the 43.8 mM formula, had a much faster time of onset inducing a bowel movement in less than three hours in all patients. Only one bisacodyl volunteer had a bowel movement in less than three hours. No study volunteer reported incontinence in association with any of the test laxatives. Gastrointestinal symptoms were similar between the 10 mg bisacodyl and the sulfate formulations and no unexpected adverse events were reported. A formulation providing a 3:1 ratio of sodium to potassium minimized sodium and potassium electrolyte absorption or loss from the laxative and was considered appropriate for further study in constipated patients at a dose of about 60 mM (5.8 grams sulfate).

A pilot study of the reformulated sulfate solution (BLI801 Laxative) was conducted to evaluate a seven day treatment of the BLI801 Laxative in adult outpatients meeting ROME III constipation criteria. The intent of this multi-center study was to determine the efficacy of the laxative as both a one day and a seven day therapy. Thirty-three patients received BLI801 laxative and nineteen received placebo (a solution of sodium chloride).

The primary endpoint of the study was the percentage of patients experiencing a bowel movement within 3 hours of the first study medication dose. The co-primary endpoint was the percentage of patients with a successful treatment week, defined as not meeting ROME III criteria at the end of the treatment week. Seventy percent of patients that received BLI801 Laxative had a bowel movement within 3 hours versus 53% of placebo patients (p=0.412). The rapid effect of BLI801 Laxative was more predictable over the treatment week, with significantly more BLI801 Laxative patients having a bowel movement within 3 hours on at least 4 out of 7 treatment days, as shown below in Table 26.

TABLE 26
Secondary Efficacy Responder Analysis Percent of Patients
having a BM within 3 hours of Dosing (min. 4 out of 7 days)

| | BLI801 | Placebo | | |
|---|---|---|---|---|
| Responder | n (%) | n (%) | 95% CI | p |
| All Patients (n) | 33 | 19 | | |
| Success | 20 (60.6%) | 4 (21.1%) | 11.4, 62.9 | 0.008 |
| Fail | 13 (39.4%) | 15 (78.9%) | | |

With respect to weekly treatment success, 69.7% of BLI801 Laxative patients no longer met the ROME III definition of constipation by the end of the study versus 26.3% of placebo recipients (p<0.01). There was no evidence of tachyphylaxis.

There were no statistically significant differences between BLI801 Laxative and placebo with respect to treatment emergent adverse events. Also, there was no significant differences seen for the expected patient reported symptoms of gas and cramping (rated using a five point scale ranging from “1=None” to “5=Severely Distressing”). More patients experienced watery stools in the BLI801 group, which is expected due to its laxative effect. There were no serious adverse events during the study and no on-study deaths. Laboratory results showed no differences between BLI801 Laxative and Placebo for serum chemistry data, including sulfate.

A second Phase 2 study was conducted to evaluate two dose levels of BLI801 Laxative taken on an as needed, or “on demand” basis for 4 weeks. Patients were instructed to take up to one full dose of BLI801 Laxative if they felt they needed relief of their constipation symptoms. Sixty constipated patients meeting ROME III criteria completed the protocol. Their average age was 43 years and study participants were predominantly female (88%).

Subjects in both the high and low dose group averaged 4 doses of BLI801 laxative during each treatment week. There was no difference between the two dose groups with respect to overall treatment success which was defined as >3 complete spontaneous bowel movements (CSBMs) during a given treatment week with an accompanying increase of at least 1 CSBM from baseline for that week for 3 out of 4 weeks of treatment. The low dose group had about 35.5% of study subjects with a successful outcome according to this definition versus 38.7% for the higher dose. When a lower success threshold was applied (2 out of 4 successful weeks), the high dose group had a success rate of 58% compared to 42% in the low dose group.

As shown in Table 27, the higher dose was associated with many more patients having a BM within three hours of dose ingestion (55%) than the lower dose (29%). This difference persisted when a more strict definition of complete, spontaneous BMs was applied.

TABLE 27
Day 1 Efficacy Parameters

| | BLI 801 | BLI801 | |
|---|---|---|---|
| | Low | High | |
| | Dose | Dose | p¹ |
| Bowel Movement (BM) | 9 (29%) | 17 (55%) | 0.062 |
| within 3 hrs | | | |
| Complete Spontaneous | 6 (19%) | 11 (36%) | 0.206 |
| BM within 3 hrs | | | |

¹P value is from a CMH Chi-square test, controlling for site

BLI801 Laxative was well tolerated in this study with few side effects other than loose stools and diarrhea. There were more reports of treatment emergent diarrhea in the high dose group (19% vs 0%). However, average Bristol stool ratings were similar between the dose groups (average scores about 4) with no effect of “on demand” dosing versus daily treatment observed. Other treatment emergent adverse events were infrequent, with no significant differences detected between the two treatments.

This study supports the conclusion that BLI801 laxative solution is effective and predictable both as a one day “on demand” treatment for chronic idiopathic constipation as well as a laxative for daily use. Research in the area of non-idiopathic constipation has been generally lacking except recently for opiate induced constipation (OIC). While it is generally accepted that opiates can be a causative factor in patients suffering from non-idiopathic constipation, there are other medication categories that are also similarly constipating. For patients taking these medications, mu opioid receptor antagonists will not be effective due to their mechanism of action. New, more versatile, treatment options are needed to provide effective constipation relief in patients taking constipating medications that cannot be treated with current OIC products. The current study will evaluate the efficacy of BLI801 Laxative in subjects whose constipation is caused by treatment with non-opiate medications.

### Example 8. Study of Solid Oral Dosage Form

Tablets were provided in three bottles containing approximately 60 tablets in each bottle:

Component Amounts
(anhydrous equivalents)

| | Na₂SO₄ | K₂SO₄ |
|---|---|---|
| Dose per 6 tablets | 5.54 g | 2.27 g |

Each bottle had a clinical label containing a caution statement, study code, study sponsor and kit number. Each subject received a 30 day supply of tablets at Visit 1. A single dose of BLI801 (maximum of six tablets) per day were taken as needed throughout the treatment period.

All study medication is required to remain at room temperature 20-25 C (68-77 F); excursions permitted between 15-30 C (59-86 F).

Subject Selection

Subjects were admitted to the study if they were: 1) Male or female subjects at least 18 years of age; 2) Taking antidepressant medication known to cause constipation; 3) Otherwise in good health, as determined by physical exam and medical history; 4) If female, and of child-bearing potential, is using an acceptable form of birth control (hormonal birth control, IUD, double-barrier method, depot contraceptive, sterilized, abstinent, or vasectomized spouse; 5) Negative urine pregnancy test at screening (visit 1), if applicable; 6) In the Investigator's judgment, subject is mentally competent to provide informed consent to participate in the study; and 7) Constipated, defined by the following criteria: i) Fewer than 3 spontaneous defecations per week and at least one of the following symptoms for the previous 4 weeks: Straining during >25% of defecations; Lumpy or hard stools in >25% of defecations; and Sensation of incomplete evacuation for >25% of defecations and ii) Onset of constipation must coincide with the instruction of treatment with a constipating medication.

Exclusion Criteria

Subjects who met any of the following criteria were excluded from the study:

1. Subjects that are taking opiates for any reason.

2. Subjects whose constipation diagnosis and symptoms predate the initiation of treatment with the constipating medication.

3. Subjects with known or suspected ileus, gastrointestinal obstruction, gastric retention, bowel perforation, toxic colitis, toxic megacolon

4. Subjects who have had major surgery 30 days before Visit 1; appendectomy or cholecystectomy 60 days before Visit 1; abdominal, pelvic, or retroperitoneal surgery 6 months before Visit 1; bariatric surgery or surgery to remove a segment of the GI tract at any time before Visit 1

5. Medical conditions associated with diarrhea, intermittent loose stools, or constipation, which could confound the interpretation of the results, eg, fecal incontinence or irritable bowel syndrome. Subjects with irritable bowel syndrome (IBS) that has been previously diagnosed by a physician prior to initiation of the constipating therapy and that meets the following criteria, are excluded: a. Absence of a structural or biochemical explanation for the abdominal pain symptom; b. At least 12 weeks during a period of 12 months, of abdominal discomfort or pain with at least 2 of the following 3 features: i. Relieved with defecation, and/or; ii. Onset associated with a change in frequency of stool, and/or iii. Onset associated with a change in form of stool.

6. Subjects taking laxatives (with the exception of fiber supplements), prokinetic agents or antidiarrheal drugs that refuse to discontinue these treatments from Visit 1 until after completion of Visit 3

7. Subjects who are pregnant or nursing, or intend to become pregnant during the study

8. Subjects of childbearing potential who refuse a pregnancy test

9. Subjects who are allergic to any BLI801 component (sodium sulfate, potassium sulfate, Polyethylene Glycol (PEG) 3350)

10. Active substance or alcohol use that, in the opinion of the investigator, could compromise patient's ability to comply with the study instructions.

11. Subjects who have participated in an investigational clinical, surgical, drug, or device study within the past 30 days

12. Subjects who, in the opinion of the Investigator, should not be included in the study for any reason, including inability to follow study procedures.

13. Subjects who withdraw consent at any time prior to completion of Visit 1 procedures

14. Subjects who have had a colonoscopy within 2 weeks of Visit 1 or are scheduled to have a colonoscopy during their participation in the study.

Study Procedures

Study procedures are described as follows. Acceptable deviations from the visit schedule are indicated.

Visit 1: Screening (Day 0)

At the screening visit, the following procedures were undertaken: Subject is fully informed about the study and gives written agreement to study participation in the form of a signed informed consent form (refer to Section 4.1.1) and assign a study number; Assess eligibility (refer to inclusion/exclusion criteria); Review of medications; Medical history including history of constipation (ROME criteria); Physical examination (including height and bodyweight); Vital signs; Urine pregnancy test (if applicable); Dispense study drug (3 bottles each containing 60 tablets) and review dosing instructions with the subject in detail to ensure full understanding; Provide the subject with a paper diary and review instructions with the subject in detail to ensure full understanding; Instruct subject to maintain their normal dietary habits during study participation; and Schedule the next study visit to occur after 14 full days.

Subjects that were ineligible due to prohibited medication use of laxatives repeated procedures and were washed out for 72 hours only. No additional procedures were performed on these patients until they completed washout. When subjects returned following washout, concomitant medications were reviewed, physical exam, vital signs, and urine pregnancy test performed (if not done previously).

Subjects were instructed to take one dose of (6 tablets) of study medication per day as needed over the next 28 days (leading up to Visit 3) if they required relief for their constipation symptoms. Dosing commenced, if needed, starting on the day after Visit 1. Subjects swallowed the tablets whole or dissolve them in 8-12 ounces of water and drank the solution. The subject reported the time of each dose, and if tablets were ingested or dissolved, in their diary. All bowel movements starting on Treatment Day 1 must be reported in the diary in real time, regardless of whether the subject takes study medication. Subjects had to enter into their medication questionnaire diary every day regardless if they took the medication or not that day.

Subjects that experience continued diarrhea and/or loose stools reduced their dose to 4 tablets. In this instance, subjects were required to notify the site personnel that they have reduced their dose.

Subject Diaries

Subjects were asked to complete a paper diary to self-report their bowel movement and medication experiences each day. Subjects were required to enter data on each bowel movement as soon as possible following completion. Each dosing episode of study medication was entered.

Visit 2-Day 14 (+/−3 days)

Subjects returned after approximately 14 days for an interim follow up visit. Study personnel reviewed the returned bottles of tablets for accountability purposes. Study personnel also reviewed the paper diaries to ensure subjects were being compliant with their completion. Study personnel discussed any diary reporting irregularities. Subjects were queried for any AE's or changes to their con meds. Study medication and paper diaries were re-dispensed.

Visit 3-Day 29 (+3 days)/Early Discontinuation/End of Treatment

Subjects returned after a full 28 days after Visit 1 for their final clinic visit. Visit 3 did not occur prior to a subject completing 28 complete consecutive days of the treatment period. Vital signs were taken. Study personnel reviewed returned bottles of tablets for accountability purposes and for consistency with the diary reporting. Study personnel discussed any diary reporting irregularities. Subjects were queried for any AE's or changes to their con meds.

Follow-up Telephone Call—Day 35 (+3 days)

At Day 35, approximately 1 week after the last study visit, or 1 week following an Early Discontinuation Visit, site personnel contacted subjects by telephone to query if any new adverse events occurred and if any adverse events ongoing at the previous visit were resolved.

Tabulated Study Procedures

The following graphically depicts the flow of study procedures at each visit.

| | | Visit 2 | Visit 3 | [symbol] |
|---|---|---|---|---|
| | | Day | Day | Day |
| | Visit | 14 +/− 3 | 29 + 3 | 35 +/− 3 |
| Procedures | 1 | days | days | days |
| Informed Consent | X | | | |
| Inclusion/Exclusion | X | | | |
| Criteria Review | | | | |
| Medical History | X | | | |
| Physical Examination | X | | X | |
| Vital Signs | X | | X | |
| Review of Concomitant | X | X | X | X |
| Medication | | | | |
| Urine Pregnancy Test | X | | | |
| (if applicable) | | | | |
| Dispense Diary | X | X | | |
| Dispense Study Drug | X | X | | |
| Study Drug and Diary | | X | X | |
| Accountability | | | | |
| Diary Accountability | | X | X | |
| Assess Safety | | X | X | X |

Concomitant Medications

The use of concomitant medication were recorded from 7 days prior to Visit 1 until completion of the Day 35 follow-up call. Subjects enrolled in this study were not permitted to take any laxatives, whether prescription or over-the-counter (with the exception of fiber supplements) from Visit 1 until after the completion of Visit 3. Medications known to effect bowel habits (refer to Section 3.5.2) were also prohibited from Visit 1 to the completion of Visit 3. Any restricted use of medications during the study resulted in termination of subject's participation.

Adverse Events

Adverse Event Definition and Reporting

An Adverse Event (AE) is any untoward medical occurrence associated with the use of a drug in humans, whether or not considered drug related. An AE can therefore be any unfavorable and unintended sign, symptom, or disease temporally associated with the use of a medicinal (investigational) product.

“Diarrhea” is defined as more than three large watery stools per day. An adverse event for “Diarrhea” should only be completed if a subject reports more than 3 bowel movements with a Bristol Stool Rating of “7” in a day.

Adverse event collection coincided with the patient providing informed consent to participate in the study and concluded with the end of study participation at the Day 35 follow-up call. Patients were instructed to report promptly adverse events to the Investigator. The Investigator recorded the date/time of report, date/time of onset, description of the adverse event, severity of adverse event, action(s) taken regarding treatment of the event, action(s) taken regarding study participation, duration of adverse event, and the Investigator's assessment of relationship of adverse event to study treatment.

The Investigator should assess the severity of each adverse event using the following categories:

The Investigator should assess the relationship to study drug for each adverse event using the following categories:

| Grade | Severity | Description |
|---|---|---|
| 1 | Mild | Barely noticeable, does not influence |
| | | functioning |
| | | Causing no limitations of usual activities |
| 2 | Moderate | Makes participant uncomfortable, influences |
| | | functioning |
| | | Causing some limitations of usual activities |
| 3 | Severe | Severe discomfort, treatment needed |
| | | Severe and undesirable, causing inability |
| | | to carry out usual activities |
| 4 | Life | Immediate risk of death |
| | threatening | Life threatening or disabling |
| 5 | Fatal | Causes death of the participant |

The Investigator should assess the relationship to study drug for each adverse event using the following categories:

| Categories of | |
|---|---|
| Attribution: | Description |
| UNRELATED | There is no evidence of any causal |
| | relationship. |
| POSSIBLE | There is some evidence to suggest a causal |
| | relationship (e.g., the event occurred within |
| | a reasonable time after administration of |
| | the trial medication). However, the influence |
| | of other factors may have contributed to the |
| | event (e.g., the patient's clinical |
| | condition, other concomitant events). |
| PROBABLE | There is evidence to suggest a causal |
| | relationship, and the influence of other |
| | factors is unlikely. |
| DEFINITE | There is clear evidence to suggest a causal |
| | relationship, and other possible contributing |
| | factors can be ruled out. |

In Phase 3 studies, adverse events associated with Oral Sodium Sulfate administered at doses consistent with colonoscopy preparation have included nausea, vomiting, abdominal cramping and bloating. These adverse reactions were transient and subsided rapidly.

In a Phase 2 study of BLI801 Laxative, the most frequently reported adverse events were abdominal pain, diarrhea, flatulence and nausea. These adverse reactions were transient and subsided rapidly.

A Serious Adverse Event (SAE) is any untoward medical occurrence that results in at least one of the following outcomes: Results in death; Is life-threatening; Requires inpatient hospitalization or prolongation of existing hospitalization; A persistent or significant incapacity or substantial disruption of the ability to conduct normal life functions; Is a congenital anomaly/birth defect; and Requires medical or surgical intervention to prevent permanent impairment or damage.

SAE collection coincided with the patient providing informed consent to participate in the study and will conclude 30 days after date of last study medication dose. Should a serious and/or unexpected adverse event occur, the Investigator must notify Braintree Laboratories immediately. The Investigator will make a decision regarding continuing study participation, and may request input from Braintree Laboratories. The Investigator will be responsible for recommending or providing the subject with appropriate medical therapy. All subjects experiencing serious adverse events will be followed until satisfactory resolution occurs. Braintree Laboratories must be kept apprised of all follow-ups relative to serious adverse events. In addition, Investigators must comply with the SAE reporting requirements of the Institutional Review Board with oversight of the study.

Data Analysis

Sample Size

For this pilot study, fifteen subjects were enrolled. The sample size was allowed for qualitative comparisons of efficacy and safety data with previous studies conducted by Braintree Laboratories using prior sulfate formulations as well as other approved laxatives. This information was used to design larger, formal studies of the sulfate laxative.

Planned Analyses

The following demographic and baseline characteristics will be presented.
Age
Gender
Race/ethnicity
Constipation History

Efficacy Analyses

Primary Efficacy Endpoint

The primary efficacy endpoint will be assessed on the basis of a binary outcome of overall treatment response. Treatment response is defined as >3 spontaneous bowel movements (SBMs) during a given treatment week. The primary endpoint is defined as treatment response during 2 out of 4 weeks of treatment. An SBM is a bowel movement that occurs with no rescue laxative use in the previous 24 hours.

Secondary Endpoints

Secondary endpoints will include the following: Modified Primary Endpoint Definition—% of subjects with treatment response in 1 of 4 weeks, 3 of 4 weeks and 4 of 4 weeks; Percent of patients with treatment response by week; Number of study medication doses taken per week (mean); % of subjects not meeting ROME criteria at the end of each treatment week; % of subjects experiencing a BM within 3 hours of the first study medication dose; Number of BMs per week (mean); Number of lumpy/hard BMs per week (mean); Number of BMs with straining per week (mean); BM urgency score per week (mean); Bristol Stool Form score per week (mean); Number of diarrhea episodes per week (mean); and Time to first BM.

Safety Analyses

Analysis of safety will be performed using the Intent to Treat population.

Adverse Events

Adverse Events will be coded using the MedDRA classification to provide a preferred term and primary system organ class for each event. Proportions of subjects with adverse events will be presented. Tables of AEs will be presented by system organ class and preferred term, and include overall totals for AEs within each system organ class. Counting will be done by subject and not by event.

Treatment-emergent AEs are defined as adverse events that had an onset day and time on or after the day and time of the first dose of study drug. Adverse Events having missing onset dates will be considered as treatment emergent.

Vital Signs

Summary statistics (i.e., mean, minimum, maximum, standard deviation, and number of subjects) will be presented for each vital sign at each visit.

Results

Nine patients were analyzed for the number of SBM per week during the treatment period. For a successful outcome, the patient had to have at least 50% of weeks during the treatment period in which the patient had at least 3 SBM per week. As shown in Table 28 below, six of the nine patients had three or more SBM in 2 of the four weeks that they participated in the study.

The disclosed formulations therefore is at least similar in its success rate to other drugs on the market. For instance, Movantik has a success rate of approximately 45%, Amitiza has a success rate of 30%, and Relistor has a success rate of 60%.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. An oral pharmaceutical composition for inducing a bowel movement in a human subject, the composition comprising sodium sulfate and potassium sulfate, wherein the composition is substantially free of magnesium sulfate, and wherein the composition does not produce clinically significant sodium and/or potassium electrolyte shifts upon administration to a human subject.

2. An oral pharmaceutical composition for treating constipation in a human subject, the composition comprising sodium sulfate and potassium sulfate, wherein the composition is substantially free of magnesium sulfate, and wherein the composition does not produce a clinically significant sodium and/or potassium electrolyte shift upon administration to a human subject.

3. The composition of claim 1 or 2, wherein the composition is suitable for administration to the human subject by direct oral ingestion.

4. The composition of claim 1 or 2, wherein the composition is suitable for administration to the human subject by disintegration in an aqueous solution prior to oral ingestion.

5. The composition of claim 1 or 2, wherein the composition is suitable for both administration to the human subject by direct oral ingestion and by disintegration in an aqueous solution prior to oral ingestion.

6. The composition of claim 1 or 2, wherein the composition is a tablet.

7. The composition of claim 1 or 2, wherein the composition is substantially free of polyethylene glycol (PEG).

8. The composition of claim 1 or 2, wherein the composition is substantially free of phosphates.

9. The composition of claim 1 or 2, wherein the composition induces a bowel movement in a human subject within 5 hours of administration.

10. The composition of claim 1 or 2, wherein the composition induces a bowel movement in a human subject within 4 hours of administration.

11. The composition of claim 1 or 2, wherein the composition induces a bowel movement in a human subject within 3 hours of administration.

12. The composition of claim 1 or 2, wherein the composition induces a bowel movement in a human subject within 2 hours of administration.

13. The composition of claim 1 or 2, wherein the composition induces 1 to 6 bowel movements in a human subject within 24 hours of administration.

14. The composition of claim 1 or 2, wherein the composition induces 1 to 3 bowel movements in a human subject within 24 hours of administration.

15. The composition of claim 1 or 2, wherein the composition induces 1 to 3 bowel movements in a human subject within 24 hours of administration.

16. The composition of claim 1 or 2, wherein the composition induces a bowel movement in a human subject with a Bristol Stool Scale rating of 4 to 7.

17. The composition of claim 1 or 2, wherein the composition comprises about 30 mmol to about 100 mmol of sulfate salts.

18. The composition of claim 17, wherein the composition comprises about 60 mmol to about 80 mmol of sulfate salts.

19. The composition of claim 17, wherein the composition comprises about 50 mmol of sulfate salts.

20. The composition of claim 17, wherein the composition comprises about 60 mmol of sulfate salts.

21. The composition of claim 17, wherein the composition comprises about 80 mmol of sulfate salts.

22. The composition of claim 1 or 2, wherein the sodium sulfate and potassium sulfate are present in the composition at a ratio of about 2:1 to about 4:1.

23. The composition of claim 1 or 2, wherein the sodium sulfate and potassium sulfate are present in the composition at a ratio of about 2.5:1 to about 3.5:1.

24. The composition of claim 1 or 2, wherein the sodium sulfate and potassium sulfate are present in the composition at a ratio of about 3:1.

25. The composition of claim 1 or 2, wherein the composition comprises about 3 g to about 10 g of sodium sulfate.

26. The composition of claim 1 or 2, wherein the composition comprises about 5 g to about 8 g of sodium sulfate.

27. The composition of claim 1 or 2, wherein the composition comprises about 1 g to about 8 g of potassium sulfate.

28. The composition of claim 1 or 2, wherein the composition comprises about 2 g to about 4 g of potassium sulfate.

29. The composition of claim 1 or 2, wherein the composition comprises about 3 g to about 10 g of total sulfate.

30. The composition of claim 1 or 2, wherein the composition comprises about 4 g of total sulfate.

31. The composition of claim 1 or 2, wherein the composition comprises about 5 g of total sulfate.

32. The composition of claim 1 or 2, wherein the composition comprises about 6 g of total sulfate.

33. The composition of claim 1 or 2, wherein the composition comprises about 7 g of total sulfate.

34. The composition of claim 1 or 2, wherein the composition comprises at least one excipient selected from the group consisting of a disintegrant, a binder, a glidant, a lubricant, a flavor, a preservative, a sweetener, and combinations thereof.

35. A method for inducing a bowel movement in a human subject, comprising orally administering to the subject a pharmaceutical composition comprising a 3:1 ratio of sodium sulfate:potassium sulfate, wherein the composition is substantially free of magnesium sulfate, and wherein the composition does not produce a clinically significant sodium and/or potassium electrolyte shift upon administration to the human subject, thereby inducing a bowel movement in the subject.

36. The method of claim 35, wherein the composition is administered to the human subject in a single dose.

37. The method of claim 36, wherein the single dose is administered once per day.

38. The method of claim 36, wherein the single dose is administered twice per day.

39. The method of claim 37, wherein the single dose is administered once per day for 1 or more days.

40. The method of claim 37, wherein the single dose is administered once per day for up to 28 days.

41. The method of claim 35, wherein the composition is administered to the subject in a single dose on each of 28 days.

42. The method of claim 35, wherein the composition induces 3 or more bowel movements within any 7 consecutive days of treatment.

43. The method of claim 35, wherein the composition induces a bowel movement in the human subject within 3 hours of administration.

44. The method of claim 35, wherein the composition induces a bowel movement in the human subject within 2 hours of administration.

45. The method of claim 35, wherein the composition induces 1 to 6 bowel movements in a human subject within 24 hours of administration.

46. A method for inducing a bowel movement in a human subject, comprising orally administering to the subject the composition of any one of claims 1-34, and allowing the composition to induce a bowel movement in the subject, thereby inducing a bowel movement in the human subject.

47. A method for treating or preventing constipation in a human subject, comprising orally administering to the subject the composition of any one of claims 1-34, in an amount effective to treat or prevent constipation in a human subject, and thereby treating or preventing constipation in the human subject.

48. The method claim 46 or 47, wherein the composition induces a bowel movement in the human subject within 5 hours of administration.

49. The method claim 46 or 47, wherein the composition induces a bowel movement in the human subject within 4 hours of administration.

50. The method claim 46 or 47, wherein the composition induces a bowel movement in the human subject within 3 hours of administration.

51. The method claim 46 or 47, wherein the human subject no longer meets the ROME III criteria following administration of the composition for 7 consecutive days.

52. The method claim 46 or 47, wherein the composition induces a bowel movement in the human subject with a Bristol Stool Scale rating of 4 to 7.

53. The method of any one of claims 35, 46, or 47, wherein the human subject does not experience a clinically significant electrolyte shift of sodium and potassium.

54. The method of any one of claim 35, 46, or 47, wherein the human subject suffers from drug-induced constipation.

55. The method of claim 54, wherein the drug-induced constipation is opioid-induced constipation.

56. The method of claim 55, wherein in the human subject is receiving opioids chronically.

57. The method of any one of claim 35, 46, or 47, wherein the human subject is a cancer patient.

58. A formulation for inducing laxation in a subject comprising from about 0.9 grams of sodium sulfate to about 8.0 grams of sodium sulfate and from about 0.3 grams to about 3.5 grams of potassium sulfate.

59. The formulation of claim 58, wherein the formulation comprises about 5.5 grams of sodium sulfate and about 2.7 grams of potassium sulfate.

60. The formulation of claim 58, wherein the formulation is a solid oral dosage form.

61. The formulation of claim 60, wherein the solid oral dosage form is selected from the group consisting of a tablet, capsule, dual purpose tablet, caplet, compressed capsule, and gel capsule.

62. The formulation of claim 60, wherein the formulation is administered as 1 to 10 tablets.

63. The formulation of claim 60, wherein the formulation is administered as 1 to 6 tablets.

64. The formulation of claim 60, wherein each solid oral dosage form comprises from about 0.5 grams to about 1.5 grams of sodium sulfate and from about 0.2 grams to about 0.6 grams of potassium sulfate.

65. The formulation of claim 60, wherein each solid oral dosage form comprises from about 0.8 grams to about 1.1 grams of sodium sulfate and from about 0.3 grams to about 0.5 grams of potassium sulfate.

66. The formulation of claim 58, wherein the formulation comprises from about 5.00 grams to about 6.00 grams of sodium sulfate and from about 2.00 grams to 2.50 grams of potassium sulfate.

67. The formulation of claim 58, wherein the formulation comprises 5.54 grams of sodium sulfate and 2.27 grams of potassium sulfate.

68. The formulation of claim 58, wherein the formulation is a powder.

69. The formulation of claim 68, wherein the formulation comprises from about 0.90 grams to about 6.00 grams of sodium sulfate and from about 0.30 grams to 2.50 grams of potassium sulfate.

70. The formulation of claim 68, wherein the formulation comprises 5.54 grams of sodium sulfate and 2.27 grams of potassium sulfate.

71. The formulation of claim 58, wherein the formulation is a liquid.

72. The formulation of claim 71, wherein the formulation comprises from about 0.90 grams to about 6.00 grams of sodium sulfate and from about 0.30 grams to 2.50 grams of potassium sulfate.

73. The formulation of claim 71, wherein the formulation comprises 5.54 grams of sodium sulfate and 2.27 grams of potassium sulfate.

74. The formulation of claim 60, wherein the formulation further comprises one or more excipients.

75. The formulation of claim 74, wherein the one or more excipients is selected from the group consisting of are selected from the group consisting of binders, lubricants, glidants, disintegrants, and combinations thereof.

76. A method of treating constipation in a subject, the method comprising:
a) administering to the subject an effective amount sodium sulfate and potassium sulfate to induce a laxative effective;
whereby the sodium sulfate and potassium sulfate cause a spontaneous bowel movement in the subject during the treatment period.

77. The method of claim 76, wherein the effective amount of sodium sulfate comprises about 0.9 grams of sodium sulfate to about 8.0 grams of sodium sulfate and from about 0.3 grams to about 3.5 grams of potassium sulfate.

78. The method of claim 76, wherein the effective amount of sodium sulfate and potassium sulfate comprises about 5.5 grams of sodium sulfate and about 2.7 grams of potassium sulfate.

79. The method of claim 76, wherein the effective amount does not cause clinically significant electrolyte shifts.

80. The method of claim 76, wherein the sodium sulfate and potassium sulfate cause at least three spontaneous bowel movements during a treatment period.

81. The method of claim 80, wherein the treatment period is seven days.

82. The method of claim 76, wherein the sodium sulfate and potassium sulfate are administered as solid oral dosage forms.

83. The method of claim 82, wherein the solid oral dosage form is selected from the group consisting of tablets, capsules, caplets, gel capsules, and compressed capsules.

84. The method of claim 83, wherein the solid oral dosage form is a tablet.

85. The method of claim 83, wherein the subject is administered 1 to 6 tablets.

86. The method of claim 85, wherein each tablet comprises from about 0.5 grams to about 1.5 grams of sodium sulfate and from about 0.2 grams to about 0.6 grams of potassium sulfate.

87. The method of claim 85, wherein each tablet comprises from about 0.8 grams to about 1.1 grams of sodium sulfate and from about 0.3 grams to about 0.5 grams of potassium sulfate.

88. The method of claim 85, wherein each tablet comprises about 0.9 grams of sodium sulfate and about 0.4 grams of potassium sulfate.

89. The method of claim 76, wherein the sodium sulfate and potassium sulfate are administered as a powder.

90. The method of claim 76, wherein the sodium sulfate and potassium sulfate are administered as a liquid.

91. The method of claim 76 further comprising administering a volume of water.

92. The method of claim 76, wherein the spontaneous bowel movement relieves a condition selected from the group consisting of bloating, gas, cramping, and combinations thereof.

93. The method of claim 76, wherein the constipation is caused by a medication.

94. The method of claim 76, wherein the constipation is caused by opioid.

95. The method of claim 76, wherein the constipation is caused by an antidepressant.
